(19) United States
(12) Patent Application Publication
(10) Pub. No.: US 2026/0317252 A1
(43) Pub. Date: Oct. 15, 2026

(54) METHOD FOR PRODUCING ALPHA-OLEFIN LOW POLYMER

(71) Applicant: Mitsubishi Chemical Corporation, Tokyo (JP)

(72) Inventors: Akihisa TOKUDA, Tokyo (JP); Takeshi MATSUO, Tokyo (JP); Jun NAGAKI, Tokyo (JP)

(73) Assignee: Mitsubishi Chemical Corporation, Tokyo (JP)

(21) Appl. No.: 19/688,804

(22) Filed: May 27, 2026

Related U.S. Application Data
(63) Continuation of application No. PCT/JP2025/033873, filed on Sep. 25, 2025.

(30) Foreign Application Priority Data
Sep. 27, 2024 (JP) 2024-168697
Sep. 27, 2024 (JP) 2024-169055

(51) Int. Cl. C07C 2/26

(52) U.S. Cl. CPC C07C 2/26; C07C 2531/12

(57) ABSTRACT

In one or some examples, a method for producing an α-olefin low polymer includes a gas drying step of supplying a gas into a reaction system, a solvent drying step of supplying a drying agent-containing solvent containing a drying agent into the reaction system after the gas drying step, a deactivator addition step of supplying a deactivator for the drying agent into the reaction system, and an α-olefin low polymerization reaction step of performing a low polymerization reaction of an α-olefin, in which the deactivator is supplied into the reaction system prior to initiation of the low polymerization reaction of the α-olefin.

The present application is a continuation of international application PCT/JP2025/033873, filed on Sep. 25, 2025, and claims priority based on JP 2024-169055 and JP 2024-168697 filed with the Japan Patent Office on Sep. 27, 2024, the contents of each of which are incorporated herein by reference.

### TECHNICAL FIELD

The present invention relates to a method for producing an α-olefin low polymer.

### BACKGROUND ART

An α-olefin low polymer is a useful substance widely used as a raw material monomer of an olefin-based polymer, as a comonomer of various polymers, and as a raw material of a plasticizer, a surfactant, a lubricating oil, and the like. Among several α-olefin low polymers, for example, 1-hexene is useful as a raw material of linear low-density polyethylene. 1-Hexene is produced by trimerization of ethylene.

An α-olefin low polymer is usually produced by subjecting a raw material α-olefin to a low polymerization reaction in the presence of a chromium-based catalyst and a reaction solvent. For example, 1-hexene is produced by a trimerization reaction of ethylene in the presence of a catalyst composition containing a transition metal-containing compound, an aluminum-containing compound, and optionally a halogen-containing compound or a nitrogen-containing compound, and a reaction solvent.

In the low polymerization reaction of an α-olefin, the presence of water in the reaction system lowers the catalytic activity, thus promoting the formation of a polymer as a by-product. When a low polymerization reaction is continued in long-term continuous operation, it is necessary to separate and remove the formed polymer. In addition, the formed polymer adheres to or deposits in the production equipment, which causes a decrease in production stability, clogging of the production equipment and other issues. Therefore, it is necessary to perform the low polymerization reaction under conditions where the moisture concentration in the system is controlled by removing moisture from the reaction system.

For example, Patent Document 1 discloses a method for adjusting the moisture content in a reactor, in which an aluminum-containing compound as one of catalyst components is introduced into a reaction system in a state where a raw material α-olefin and a reaction solvent are circulated in advance before the catalyst components for a low polymerization reaction are supplied into the reaction system.

Patent Document 2 discloses a method for adjusting the moisture content in a reactor, in which a solvent to be supplied to a reaction system is distilled and purified in advance and then supplied to the reaction system. Patent Document 2 also discloses that a solvent is purified in advance using an aluminum-containing compound, which is one of catalyst components, as a drying agent, and then the solvent is supplied into the reaction system.

### CITATION LIST

### Patent Literature

Patent Document 1: JP 2011-219474 A
Patent Document 2: JP H08-239330 A

### SUMMARY OF INVENTION

### Technical Problem

However, in each of the methods of Patent Documents 1 and 2, there is room for rationalization in an industrial scale production process of an α-olefin low polymer. In particular, a considerable amount of time is required to reduce the moisture content in the reactor to a desired level prior to initiation of the low polymerization reaction of the α-olefin. Therefore, the low polymerization reaction of the α-olefin is not rapidly initiated.

In addition, reduction in the material cost is important from the viewpoint of production cost and the like. In this regard, the method described in Patent Document 1 may cause an unnecessary increase in raw material cost. In addition, the load of the waste liquid treatment after the moisture reduction treatment may increase. This leads to an unnecessary increase in production cost. The method described in Patent Document 2 may cause an unnecessary increase in purification cost.

An object of the present invention is to provide an industrially advantageous method for producing an α-olefin low polymer, which can efficiently reduce the moisture content in the reaction system while suppressing the amount of an expensive alkylaluminum compound used as a drying agent in the production of an α-olefin low polymer, can start the production operation promptly, can maintain the catalytic activity and the product selectivity well when the low polymerization reaction of an α-olefin is initiated, and can also suppress the formation of a polymeric by-product that causes process blockage.

### Solution to Problem

Preferred embodiments include, but are not limited to, the following:
[1] A method for producing an α-olefin low polymer, the method including:
a gas drying step of supplying a gas into a reaction system;
a solvent drying step of supplying a drying agent-containing solvent containing a drying agent into the reaction system after the gas drying step;
a deactivator addition step of supplying a deactivator for the drying agent into the reaction system; and
an α-olefin low polymerization reaction step of performing a low polymerization reaction of an α-olefin, in which the deactivator is supplied into the reaction system prior to initiation of the low polymerization reaction of the α-olefin.
[2] The method according to [1], in which, in the gas drying step, the gas is supplied into the reaction system until a moisture content in the reaction system becomes equal to or less than a predetermined value (1).
[3] The method according to [2], in which the predetermined value (1) is 100 mol ppm.
[4] The method according to any one of [1] to [3], in which a moisture content of the gas is 5 mol ppm or less.
[5] The method according to any one of [1] to [4], in which, in the solvent drying step, the drying agent-containing solvent is supplied into the reaction system until a moisture content in the reaction system becomes equal to or less than a predetermined value (2).
[6] The method according to [5], in which the predetermined value (2) is 10 mol ppm.
[7] The method according to any one of [1] to [6], in which the drying agent-containing solvent contains an alkylaluminum compound.
[8] The method according to any one of [1] to [7], in which the deactivator contains an alkyl alcohol.
[9] The method according to any one of [1] to [8], further including, after the gas drying step and before the solvent drying step, a raw material replacement step of supplying a raw material α-olefin into the reaction system until a nitrogen gas content in the reaction system becomes equal to or less than a predetermined value (3).
[10] The method according to [9], in which the predetermined value (3) is 20 vol %.
[11] The method according to [9], further including, after the raw material replacement step and before the solvent drying step, a solvent replacement step of supplying a reaction solvent into the reaction system until a content of the α-olefin in the reaction system becomes equal to or less than a predetermined value (4).
[12] The method according to [11], in which the predetermined value (4) is 70 vol %.
[13] The method according to any one of [1] to [12], further including, after the deactivator addition step and before the α-olefin low polymerization reaction step, a measurement step of measuring a moisture content in the reaction system,
in which the α-olefin low polymerization reaction step is started when the moisture content measured in the measurement step is equal to or less than a predetermined value (5).
[14] The method according to [13], in which the predetermined value (5) is 10 mol ppm.
[15] The method according to any one of [1] to [14], in which, in the α-olefin low polymerization reaction step, a catalyst, a solvent, and a raw material α-olefin are supplied into the reaction system.

### Advantageous Effects of Invention

According to the present invention, in producing an α-olefin low polymer by a low polymerization reaction of an α-olefin, the moisture content in the reaction system is efficiently reduced prior to the start of the production operation while suppressing the amount of an expensive alkylaluminum compound used as a drying agent, thus enabling the production operation to be started promptly. Furthermore, when the low polymerization reaction of α-olefin is initiated, the catalytic activity and the product selectivity are maintained well, and the formation of a polymeric by-product that causes process blockage is also suppressed.

### BRIEF DESCRIPTION OF DRAWINGS

FIG. 1 is a schematic diagram of an example of an apparatus for producing an α-olefin low polymer (1-hexene).

FIG. 2 is a graph showing the relationship between the H₂O/Cr molar ratio and the catalytic activity value obtained in Reference Experimental Example 1.

FIG. 3 is a graph showing the relationship between the H₂O/Cr molar ratio and the polyethylene selectivity obtained in Reference Experimental Example 1.

FIG. 4 is a graph showing the relationship between the molar ratio of the deactivator to the drying agent (TEA) and the isomerization rate of 1-hexene, obtained in Reference Experimental Example 4.

### DESCRIPTION OF EMBODIMENTS

In the present specification, a numerical range indicated using “(from) . . . to . . . ” includes, unless otherwise specified, the former number as the lower limit value and the latter number as the upper limit value. “From A to B” means A or more and B or less.

In the present specification, “including A or B” means “including A”, “including B”, and “including A and B” unless otherwise specified.

In the present specification, “mass %” indicates a content proportion of a predetermined component contained in 100 mass % of the total amount. The terms “mass %” and “wt. %” are synonymous.

In the present specification, “mass ppm” refers to “ppm” calculated using a unit of mass and indicates a content proportion of a predetermined component contained in 1000000 mass ppm of the total amount. In addition, 1 mass ppm means 1×10⁻⁴ mass %. The terms “mass ppm” and “weight ppm” are synonymous.

In the present specification, “mol %” indicates a molar concentration of a predetermined component contained in the total volume.

In the present specification, “mol ppm” refers to “ppm” calculated using a unit as a mole and indicates a molar concentration of a predetermined component contained in the total volume in parts per million.

In the present specification, “optional” or “optionally” means that the circumstance described following these words may or may not occur. The description includes a case where the situation occurs and a case where the situation does not occur.

All processes described in the present description may be performed in any suitable order unless otherwise noted in the present description or unless clearly contradicted by the context.

Embodiments of the present invention will be described in detail below; however, the present invention is not limited to the following description, which relates to some examples of embodiments of the present invention.

### Method for Producing α-Olefin Low Polymer

The method for producing an α-olefin low polymer of the present invention relates to a method for producing an α-olefin low polymer by a low polymerization reaction of an α-olefin.

The method for producing an α-olefin low polymer of the present invention includes a gas drying step of supplying a gas into a reaction system, a solvent drying step of supplying a drying agent-containing solvent containing a drying agent into the reaction system after the gas drying step, a deactivator addition step of supplying a deactivator for the drying agent into the reaction system, and an α-olefin low polymerization reaction step (hereinafter sometimes simply referred to as “low polymerization reaction step”) of performing a low polymerization reaction of an α-olefin, in which the deactivator is supplied into the reaction system prior to initiation of the low polymerization reaction of the α-olefin.

Furthermore, the method for producing an α-olefin low polymer of the present invention may further include, after the deactivator addition step and before the α-olefin low polymerization reaction step, a measurement step described later of measuring the moisture content in the reaction system, and the α-olefin low polymerization reaction step may be started when the moisture content measured in the measurement step is equal to or less than a predetermined value (5).

The method for producing an α-olefin low polymer of the present invention includes, before the α-olefin low polymerization reaction step, first reducing the moisture content in the reaction system to a certain level in a gas drying step, and then reducing the moisture content to a desired value by performing a solvent drying step.

Furthermore, the method for producing an α-olefin low polymer of the present invention includes performing a deactivator addition step prior to initiation of the low polymerization reaction of the α-olefin to deactivate the drying agent such as an alkylaluminum compound supplied in the solvent drying step.

In the method for producing an α-olefin low polymer of the present invention, the gas drying step, the solvent drying step, and the deactivator addition step are performed in combination in this order at the start of operation of the apparatus for producing an α-olefin low polymer, particularly before the low polymerization reaction of the α-olefin, and the moisture content in the reaction system is efficiently reduced while suppressing the amount of the drying agent used, and the production operation may be started quickly.

Furthermore, in the method for producing an α-olefin low polymer of the present invention, by performing the deactivator addition step, when the low polymerization reaction of an α-olefin is initiated at the beginning of the operation, the catalytic activity and the product selectivity are maintained well, and the formation of a polymeric by-product that causes process blockage may also be suppressed. In particular, in the case of using an expensive alkylaluminum compound as a drying agent, the amount of the compound used may be suppressed, and thus the economic advantage may be enhanced.

In the method for producing an α-olefin low polymer of the invention, after the moisture content in the reaction system is found to be reduced to a desired value in the measurement step, the low polymerization reaction step is performed, and the formation of a polymer as a by-product that causes process blockage may also be suppressed.

In the present invention, by performing the gas drying step before performing the solvent drying step, it is possible to reduce the amount of the drying agent, particularly the expensive alkylaluminum compound used as the drying agent, used in the solvent drying step. As a result, an unnecessary increase in raw material cost may be suppressed. In addition, the moisture content in the reaction system may be reduced in a shorter time than in the known method, and therefore, the production of the α-olefin low polymer may be started promptly. As described above, in the present invention, by performing the gas drying step before the solvent drying step, the reduction of the moisture content in the solvent drying step may be more efficiently performed.

The method for producing an α-olefin low polymer of the present invention further includes a low polymerization reaction step of an α-olefin described later, in which the α-olefin low polymer is produced by supplying the raw material α-olefin, the solvent, and the catalyst into the reaction system after the deactivator addition step. Details of the α-olefin low polymerization reaction step will be described later.

The method for producing an α-olefin low polymer of the present invention further includes a deactivator addition step described later, in which a deactivator for the drying agent is supplied into the reaction system from a deactivator supply unit provided downstream of the supply unit for the drying agent-containing solvent prior to initiation of the low polymerization reaction of the α-olefin.

Furthermore, the method for producing an α-olefin low polymer of the invention may include, after the gas drying step and before the solvent drying step, a raw material replacement step described later, in which a raw material α-olefin is supplied into the reaction system until a nitrogen gas content in the reaction system becomes equal to or less than a predetermined value (3).

The method for producing an α-olefin low polymer of the invention may further include, after the raw material replacement step and before the solvent drying step, a solvent replacement step described later, in which a reaction solvent is supplied into the reaction system until a content of the α-olefin in the reaction system becomes equal to or less than a predetermined value (4).

In the present specification, the term “in the reaction system” refers to the inside of production equipment in which raw materials and reaction products used for producing an α-olefin low polymer stay or circulate. In the specific embodiment described above, the inside of the production equipment used for performing the measurement step, the low polymerization reaction step, the gas drying step, the solvent drying step, the deactivator addition step, the raw material replacement step, and the solvent replacement step corresponds to the inside of the reaction system.

Each step of the method for producing an α-olefin low polymer of the present invention will be described in detail below; however, the present invention is not limited to the following description, which relates to some examples of embodiments.

### Gas Drying Step

In the gas drying step, a gas is supplied into the reaction system prior to the low polymerization reaction of the α-olefin.

By performing the gas drying step, the inside of the reaction system is dried prior to the low polymerization reaction of the α-olefin, thereby reducing the moisture content in the reactor before the start of production operation. Consequently, the moisture content in the reaction system is efficiently reduced while suppressing the amount of drying agent used, thereby enabling the production operation to be started promptly. Furthermore, during the low polymerization of α-olefin, the catalytic activity and the product selectivity are maintained well, and the formation of a polymeric by-product that causes process blockage is more effectively suppressed. The formation of a polymeric by-product causing process blockage may be more effectively suppressed.

In particular, in the case of using an expensive alkylaluminum compound as a drying agent, the amount of the compound used is suppressed, and thus the economic advantage may be enhanced.

The gas preferably contains no oxygen in order to suppress deterioration of the catalyst in the subsequent low polymerization reaction step. The gas is preferably at least one inert gas selected from the group consisting of nitrogen gas, helium gas, neon gas, and argon gas, and is preferably nitrogen gas from the viewpoint of production cost.

### Dry Gas

As the gas, a dry gas is used for the purpose of drying the inside of the reaction system. As the dry gas, for example, a gas dried in advance with a drying agent such as a molecular sieve may be used.

The upper limit of the moisture content of the drying gas is not particularly limited, and is preferably 5 mol ppm or less, more preferably 4 mol ppm or less, still more preferably 3 mol ppm or less, and particularly preferably 2 mol ppm or less, with respect to the total amount of the drying gas, from the viewpoint of reducing the amount of drying agent, particularly the drying agent used in the solvent drying step, and further reducing the moisture content in the reaction system in a short time to promptly start the production operation of the α-olefin low polymer. In particular, in the case of using an expensive alkylaluminum compound as a drying agent, the amount of the compound used is suppressed, and thus the economic advantage may be enhanced.

The lower limit of the moisture content of the dry gas is not particularly limited, and may be usually 0.0001 mol ppm or more, more preferably 0.001 mol ppm or more, still more preferably 0.01 mol ppm or more, and particularly preferably 0.1 mol ppm or more from the viewpoint of economic efficiency such as production cost required to reduce the moisture content in the dry nitrogen.

The above upper and lower limits may be combined in any manner. For example, the moisture content of the dry gas is not particularly limited, and may be 0.0001 mol ppm or more and 5 mol ppm or less, more preferably 0.001 mol ppm or more and 4 mol ppm or less, still more preferably 0.01 mol ppm or more and 3 mol ppm or less, and particularly preferably 0.1 mol ppm or more and 2 mol ppm or less.

The moisture concentration in the dry gas is accurately measured using a known analysis method with a dew point meter. Specifically, the dew point is calculated under atmospheric pressure conditions based on the relative humidity measured in accordance with JIS Z 8806-2001.

Examples of the method for adjusting the moisture content of the dry gas include, but are not particularly limited to, known moisture removal methods such as a method for removing moisture by passing nitrogen gas through a drying agent and a method for condensing moisture in the gas by cooling nitrogen gas.

### Predetermined Value (1)

The predetermined value (1) is an upper limit allowable value of the moisture content when the moisture content in the reaction system is reduced in the gas drying step. In the gas drying step, the gas is preferably supplied into the reaction system until the moisture content in the reaction system becomes equal to or less than the predetermined value (1). The predetermined value (1) is an upper limit allowable value when the moisture content in the reaction system is reduced in the gas drying step. The predetermined value (1) is a value within a range in which the moisture content in the reaction system during production of the α-olefin low polymer does not cause a practical problem.

The predetermined value (1) may be, for example, a value empirically determined using a production apparatus or the like to be used or a value theoretically derived using simulation or the like.

For the moisture content and the supply amount of the gas required to adjust the moisture content in the reaction system in the gas drying step to the predetermined value (1) or less, optimal values may be determined based on the internal volume and residence time of the reaction system, the initial moisture content in the reactor, the reaction temperature in the reactor, and the like.

The moisture content and the supply amount of the dry nitrogen gas may be grasped in advance by experiments so that the moisture content in the reaction system becomes equal to or less than the predetermined value (1) according to the combination of the production conditions adopted when performing the gas drying step. These values may also be calculated by simulation. In experimental examples described later, an experimental value obtained from the experimental results of an appropriate number of trials using the production equipment of the α-olefin low polymer actually used was used as the predetermined value (1).

The predetermined value (1) is not particularly limited, and from the viewpoint of reducing the amount of drying agent used in the solvent drying step and further reducing the moisture content in the reaction system in a short time to promptly start the production operation of the α-olefin low polymer, the predetermined value (1) is preferably 100 mol ppm, more preferably 20 mol ppm, still more preferably 10 mol ppm, even more preferably 5 mol ppm, further more preferably 4 mol ppm, particularly preferably 3 mol ppm, and most preferably 2 mol ppm, with respect to the total amount of the gas in the reaction system.

In particular, in the case of using an expensive alkylaluminum compound as a drying agent, the amount of the compound used is suppressed, and thus the economic advantage may be enhanced.

The lower limit of the moisture content in the reaction system in the gas drying step is not particularly limited, and may be usually 0.0001 mol ppm, more preferably 0.0003 mol ppm, still more preferably 0.001 mol ppm, even more preferably 0.003 mol ppm, further more preferably 0.01 mol ppm, particularly preferably 0.03 mol ppm, and most preferably 0.1 mol ppm, from the viewpoint of economic efficiency such as an increase in production cost required to reduce the moisture content in the reaction system.

The upper limit values and the lower limit values may be optionally combined.

The moisture content in the reaction system in the gas drying step may be measured using a known moisture content analyzer. The moisture content analyzer may be an offline type or an online type provided at any position in the reaction system. For the moisture content analysis, for example, a moisture content analyzer of an electric capacity type, an electric resistance type, a microwave type, a near-infrared ray type, a neutron type, or the like may be used.

### Solvent Drying Step

In the solvent drying step, a drying agent-containing solvent described later is supplied from a supply unit into the reaction system after the gas drying step and before the α-olefin low polymerization reaction step.

By performing the solvent drying step, the inside of the reaction system after the gas drying step is further dried, and the moisture content in the reactor is further reduced before the low polymerization reaction of the α-olefin. Consequently, the moisture content in the reaction system is more efficiently reduced, thereby enabling the production operation to be started promptly. Furthermore, when the low polymerization reaction of α-olefin is initiated, the catalytic activity and the product selectivity are maintained well, and the formation of a polymeric by-product that causes process blockage may also be suppressed more effectively.

### Drying Agent-Containing Solvent

The drying agent-containing solvent used in the solvent drying step refers to a solvent containing a drying agent described later.

The drying agent is not particularly limited as long as it is a compound having an effect of substantially reducing water in the system by reacting with water to take water into the molecule or adsorbing water into the drying agent, or a compound having a hygroscopic effect.

### Drying Agent

Examples of the drying agent include an aluminum compound, an alkylaluminum compound, silica gel, a calcium salt, a molecular sieve, phosphoric pentoxide, and sodium sulfate. Among these, an alkylaluminum compound is preferred because it has excellent performance of removing water in the reaction system and excellent solubility in a reaction solvent used for the low polymerization reaction of an α-olefin, and has a catalytic action of the low polymerization reaction of an α-olefin. That is, the drying agent-containing solvent preferably contains an alkylaluminum compound as a drying agent. Details of the alkylaluminum compound will be described later.

### Solvent

The solvent is not particularly limited, and for example, a reaction solvent used for a low polymerization reaction of an α-olefin may be used.

The reaction solvent is not particularly limited, and a saturated hydrocarbon is preferably used. Examples of the reaction solvent include chain saturated hydrocarbons having from 1 to 20 carbon atoms or alicyclic saturated hydrocarbons having from 1 to 20 carbon atoms, such as butane, pentane, 3-methylpentane, n-hexane, n-heptane, 2-methylhexane, octane, cyclohexane, methylcyclohexane, 2,2,4-trimethylpentane, and decalin; and aromatic hydrocarbons such as benzene, toluene, xylene, ethylbenzene, mesitylene, and tetralin. One of the reaction solvents may be used alone, or two or more thereof may be used in combination. Among these reaction solvents, chain saturated hydrocarbons and alicyclic saturated hydrocarbons having from 4 to 10 carbon atoms are preferred from the viewpoint of suppressing the formation or precipitation of by-product polymers such as polyethylene and further from the viewpoint of high catalytic activity. Non-limiting examples thereof preferably include n-heptane and cyclohexane, and more preferably n-heptane.

The lower limit of the content proportion of the drying agent contained in the drying agent-containing solvent is not particularly limited, and is usually preferably 0.000001 wt. % or more, more preferably 0.0001 wt. % or more, and still more preferably 0.0001 wt. % or more, with respect to the total mass of the drying agent-containing solvent. The upper limit of the content proportion of the drying agent is not particularly limited, and is usually preferably 10 wt. % or less, more preferably 1 wt. % or less, and still more preferably 0.1 wt. % or less.

The above upper and lower limits may be combined in any manner. The content proportion of the drying agent contained in the drying agent-containing solvent is not particularly limited, and is, for example, usually preferably 0.000001 wt. % or more and 10 wt. % or less, more preferably 0.0001 wt. % or more and 1 wt. % or less, and still more preferably 0.0001 wt. % or more and 0.1 wt. % or less.

### Alkylaluminum Compound

The alkylaluminum compound used as the drying agent is not particularly limited as long as it is a compound in which 1 or more and 3 or less alkyl groups are bonded to an aluminum atom. From the viewpoint of excellent decomposition stability of the alkylaluminum compound in the reaction system and excellent performance of removing water in the reaction system, a linear alkylaluminum compound in which at least one of the alkyl groups directly bonded to the aluminum atom is a linear alkyl group is preferred, and a linear alkylaluminum compound in which all of the alkyl groups directly bonded to the aluminum atom are linear alkyl groups is more preferred.

Examples of the alkylaluminum compound include trialkylaluminum, alkylaluminum halide, alkoxyalkylaluminum, and alkylaluminum hydride.

Examples of the trialkylaluminum include trimethylaluminum, triethylaluminum, and triisobutylaluminum.

Examples of the alkylaluminum halide include diethylaluminum monochloride, ethylaluminum sesquichloride, and ethylaluminum dichloride.

Examples of the alkoxyalkylaluminum include diethylaluminum ethoxide.

Examples of the alkylaluminum hydride include diethylaluminum hydride.

Among the alkylaluminum compounds, trialkylaluminum is preferred, and triethylaluminum is more preferred.

One of the alkylaluminum compounds may be used alone, or two or more thereof may be used in combination.

In the present invention, by performing the solvent drying step after the gas drying step, the moisture content present in the reaction system is more efficiently reduced by the alkylaluminum compound. Since the moisture content in the reaction system is reduced to some extent after the gas drying step, the moisture content in the reaction system is more efficiently reduced while suppressing the amount of the expensive alkylaluminum compound used by performing the alkylaluminum compound solvent drying step after the gas drying step.

For the above reasons, when the low polymerization reaction of α-olefin is initiated, the catalytic activity and the product selectivity are maintained well, and the formation of a polymeric by-product that causes process blockage may also be suppressed.

The reason why the moisture content in the reaction system is reduced by the alkylaluminum compound is not clear, but is presumed as follows.

Since the alkylaluminum compound has high reactivity with water, the alkyl group thereof is substituted for a hydrogen atom of water, and an aluminum compound containing a hydroxyl group or an oxygen atom and an alkane are produced. For example, in the case of trialkylaluminum represented by (R)₃Al, the following reaction occurs.

(R)₃Al+H₂O→R₂AlOH+RH
where R is any alkyl group.

The hydroxyalkylaluminum compound (R₂AlOH) further reacts with water. At this time, various chemical species such as RAl(OH)₂, Al(OH)₃, R₂Al—O—AlR₂, R—Al(OH)—O—Al—R(OH), (—AlRO-)n, Al₂O₃ are formed.

Through the above reaction, the water in the reaction system is taken into a part of the aluminum compound. Therefore, it is presumed that the moisture content in the reaction system may be reduced by discharging these aluminum compounds out of the reaction system.

The reason why the formation of a polymeric by-product is suppressed by reducing the moisture content in the reaction system is not clear, but is presumed as follows.

When water is present in the reaction system, an aluminum-containing compound (c), which is one of the catalyst components described below, may produce a hydroxide or an aluminum polymer. As a result, it is presumed that coordination inhibition between the aluminum-containing compound (c) and the transition metal-containing compound (a) as another catalyst component, and coordination inhibition between the aluminum-containing compound (c) and the halogen-containing compound (d) as another catalyst component occur. At this time, a catalyst species different from the intended catalyst species may be produced, and therefore, the amount of a polymer as a by-product formed is promoted. It is presumed that the formation of a polymeric by-product may be suppressed by reducing the moisture content in the reaction system.

The upper limit of the temperature in the reaction system when the solvent drying step is performed is not particularly limited, and may be usually 140° C. or less, more preferably 120° C. or less, and still more preferably 100° C. or less. Setting the temperature to 140° C. or less may suppress thermal decomposition of the drying agent, and the dehydration performance may be maintained. In particular, when an alkylaluminum compound is used as the drying agent, thermal decomposition may be suppressed by setting the temperature in the reaction system to 140° C. or less. The lower limit of the temperature in the reaction system is not particularly limited, and may be usually 0° C. or more, more preferably 20° C. or more, and still more preferably 50° C. or more. When the temperature is 0° C. or more, the dehydration rate may be maintained at a favorable level without suppressing the reactivity between the alkylaluminum compound and water.

The above upper and lower limits may be combined in any manner. The temperature in the reaction system in the solvent drying step is not particularly limited, and may be usually 0° C. or more and 140° C. or less, more preferably 20° C. or more and 120° C. or less, and still more preferably 50° C. or more and 100° C. or less.

In the drying agent drying step, the method for supplying the alkylaluminum compound is not particularly limited. A method for supplying the compound as a solid or a method for supplying the compound as a solution in which the compound is dissolved in a reaction solvent described later may be used. From the viewpoint of efficiently removing water in the reaction system, it is preferable to supply the alkylaluminum compound as a solution in which the alkylaluminum compound is dissolved in the reaction solvent.

### Predetermined Value (2)

In the solvent drying step, the drying agent-containing solvent is preferably supplied into the reaction system until the moisture content in the reaction system becomes equal to or less than the predetermined value (2).

The predetermined value (2) is an upper limit allowable value when the moisture content in the reaction system is reduced in the solvent drying step. The predetermined value (2) is a value within a range in which the moisture content in the reaction system during production of the α-olefin low polymer does not cause a practical problem.

The predetermined value (2) may be, for example, a value empirically determined using a production apparatus or the like to be used or a value theoretically derived using simulation or the like.

For the supply amount of the alkylaluminum compound required to adjust the moisture content in the reaction system in the solvent drying step to the predetermined value (2) or less, an optimal value is determined based on the internal volume and residence time of the reactor, the moisture content in the reactor, the reaction temperature in the reactor, and the like.

The amount of the drying agent such as an alkylaluminum compound to be supplied may be determined in advance by experiments so that the moisture content in the reaction system becomes equal to or less than the predetermined value (2) according to the combination of the production conditions adopted when performing the solvent drying step. These values may also be calculated by simulation. In experimental examples described later, an experimental value obtained from the experimental results of an appropriate number of trials using the production equipment of the α-olefin low polymer actually used was used as the predetermined value (2).

The predetermined value (2) is not particularly limited, and from the viewpoint of maintaining the catalytic activity and the product selectivity well and suppressing the formation of a polymeric by-product that causes process blockage when the low polymerization reaction of an α-olefin is initiated, the predetermined value (2) is preferably 10 mol ppm, more preferably 5.0 mol ppm, still more preferably 3.0 mol ppm, particularly preferably 1.0 mol ppm, and most preferably 0.5 mol ppm.

The lower limit value of the moisture content in the reaction system in the solvent drying step is not particularly limited, and is usually preferably 0.0001 mol ppm, more preferably 0.001 mol ppm, still more preferably 0.01 mol ppm, particularly preferably 0.1 mol ppm, and most preferably 0.3 mol ppm, from the viewpoint of economic efficiency such as an increase in production cost required to reduce the moisture content in the reaction system.

The moisture content in the reaction system in the solvent drying step may be measured using a known moisture content analyzer. The moisture content analyzer may be an offline type or an online type provided at any position in the reaction system. For the moisture content analysis, for example, a moisture content analyzer of an electric capacity type, an electric resistance type, a microwave type, a near-infrared ray type, a neutron type, or the like may be used.

### Deactivator Addition Step

In the deactivator addition step, the deactivator of the drying agent is supplied into the reaction system.

As a method for supplying the deactivator, when a drying agent, particularly an alkylaluminum compound, is used as the drying agent, the deactivator for the drying agent may be supplied from a deactivator supply unit provided downstream of the supply unit for the drying agent-containing solvent.

The deactivator may be supplied into the reaction system prior to initiation of the low polymerization reaction of the α-olefin, and the time point of the start of the deactivator addition step is not particularly limited. The deactivator addition step may be started after the start of the solvent drying step, may be started before the start of the solvent drying step, or may be started simultaneously with the solvent drying step.

When the drying agent such as an alkylaluminum compound supplied in the solvent drying step is present in the reaction system downstream of the reactor, the low polymerization reaction of the α-olefin may continue at the start of the low polymerization reaction step described later, and by-products may be formed to decrease the product selectivity.

In the method for producing an α-olefin low polymer of the present invention, by performing the deactivator addition step, low polymerization of the α-olefin after the reactor is suppressed, and a decrease in product selectivity due to the formation of a by-product and the formation of a polymeric by-product that causes process blockage is more effectively suppressed.

Examples of the deactivator include, but are not particularly limited to, alkyl alcohols such as 2-ethylhexanol (2EH), methanol, ethanol, propanol, 1-butanol, 2-butanol, isobutanol, 1-pentanol, 2-pentanol, 3-pentanol, 1-hexanol, 2-hexanol, 3-hexanol, and 1-octanol, and water when an alkylaluminum compound is used as a drying agent. The alkyl alcohol is preferably substituted for the alkyl group of the alkylaluminum compound because the alkylaluminum compound is efficiently deactivated.

Examples of the method for supplying the raw material deactivator into the reaction system include, but are not particularly limited to, a method for supplying the alkyl alcohol itself to the reaction solvent from an appropriate position in the reaction system, and a method for supplying a mixed solution with the reaction solvent containing the alkyl alcohol in a range of 1 to 99 mass %.

The supply amount of the deactivator in the deactivator addition step is not particularly limited as long as the alkylaluminum compound used as a drying agent is completely deactivated. The supply amount may be usually 1.5 times or more the amount of the alkylaluminum compound completely deactivated. The supply amount is preferably twice or more, more preferably three times or more, the amount of the alkylaluminum compound completely deactivated.

### Raw Material Replacement Step

In the raw material replacement step, the raw material α-olefin is supplied into the reaction system after the gas drying step and before the solvent drying step until a nitrogen gas content in the reaction system becomes equal to or less than a predetermined value (3).

When nitrogen gas is selected in the gas drying step, nitrogen gas may remain in the reaction system at the stage of reaching the low polymerization reaction step described later. When the low polymerization reaction step is carried out in a state where nitrogen gas remains in the reaction system, nitrogen gas accumulates in the reactor during the reaction, and as a result, the gas partial pressure of the raw material α-olefin decreases, and the low polymerization reaction rate of the α-olefin decreases. By performing the raw material replacement step after the gas drying step, accumulation of nitrogen gas in the reactor during the low polymerization reaction step is prevented, and therefore, the productivity of the α-olefin low polymer may be more effectively maintained.

Examples of the method for supplying the raw material α-olefin into the reaction system include, but are not particularly limited to a method for supplying a single substance of the raw material α-olefin in a liquid state or a gaseous state, and a method for supplying the raw material α-olefin as a mixed solution with a reaction solvent containing the raw material α-olefin in a range of 1 to 50 mass %. From the viewpoint of recycling the supplied raw material α-olefin, a method for supplying the raw material α-olefin in a gaseous state is preferred.

### Predetermined Value (3)

The predetermined value (3) is an upper limit allowable value of the nitrogen gas content when the nitrogen gas content in the reaction system is reduced in the raw material replacement step. In the raw material replacement step, the raw material α-olefin is preferably supplied into the reaction system until the nitrogen gas content in the reaction system becomes equal to or less than the predetermined value (3). The predetermined value (3) is a value within a range in which the nitrogen gas content in the reaction system in the raw material replacement step during production of the α-olefin low polymer does not cause a practical problem.

The predetermined value (3) may be, for example, a value empirically determined using a production apparatus or the like to be used or a value theoretically derived using simulation or the like.

For the supply amount of the raw material α-olefin required to adjust the nitrogen gas content in the reaction system in the raw material replacement step to the predetermined value (3) or less, an optimum value may be determined based on the internal volume and residence time of the reactor, the moisture content in the reactor, the reaction temperature in the reactor, and the like.

The supply amount of the raw material α-olefin may be grasped in advance by experiments so that the nitrogen gas content in the reaction system becomes equal to or less than the predetermined value according to the combination of the production conditions adopted when performing the raw material replacement step. These values may also be calculated by simulation. In experimental examples described later, an experimental value obtained from the experimental results of an appropriate number of trials using the production equipment of the α-olefin low polymer actually used was used as the predetermined value (3).

The predetermined value (3) is not particularly limited, and when the raw material α-olefin is in a gas state, the predetermined value (3) is preferably 20 vol %, more preferably 10 vol %, still more preferably 5 vol %, even more preferably 3 vol %, particularly preferably 1 vol %, and most preferably 0.5 vol %, with respect to 100% by total volume of the raw material α-olefin, from the viewpoint of maintaining good productivity of the α-olefin low polymer while suppressing accumulation of residual nitrogen gas in the reactor.

The lower limit value of the nitrogen gas content in the reaction system in the raw material replacement step is not particularly limited, and from the viewpoint of economic efficiency such as an increase in production cost required to reduce the nitrogen gas content in the reaction system, the lower limit value may be usually set to 0.0001 vol %, more preferably 0.0003 vol %, still more preferably 0.001 vol %, even more preferably 0.003 vol %, particularly preferably 0.01 vol %, and most preferably 0.1 vol %.

### Solvent Replacement Step

In the solvent replacement step, the reaction solvent is supplied into the reaction system after the raw material replacement step and before the solvent drying step until the content of the α-olefin in the reaction system becomes equal to or less than a predetermined value (4).

The inside of the reaction system immediately after the raw material replacement step is in a state of being filled with the α-olefin gas. Even if the alkylaluminum compound is supplied to the reaction system in this state, a sufficient drying effect may not be achieved. By performing the solvent replacement step and filling the reaction system with the reaction solvent, the drying treatment with the alkylaluminum compound is more efficiently performed.

The supply amount of the reaction solvent in the solvent replacement step is not particularly limited as long as the reaction system is substantially filled with the reaction solvent, and may be usually 1.5 times or more, more preferably 2 times or more, still more preferably 3 times or more the total volume to be subjected to solvent replacement.

### Predetermined Value (4)

The predetermined value (4) is an upper limit allowable value of the content of the α-olefin when the α-olefin content in the reaction system is reduced in the solvent replacement step. In the solvent replacement step, the reaction solvent is preferably supplied into the reaction system until the α-olefin content in the reaction system becomes equal to or less than the predetermined value (4). The predetermined value (4) is a value within a range in which the α-olefin content in the reaction system in the solvent replacement step during production of the α-olefin low polymer does not cause a practical problem.

The predetermined value (4) may be, for example, a value empirically determined using a production apparatus or the like to be used or a value theoretically derived using simulation or the like.

The predetermined value (4) is not particularly limited, and from the viewpoint of more efficiently performing the drying treatment with a drying agent such as an alkylaluminum compound, the predetermined value (4) is preferably 70 vol %, more preferably 65 vol %, still more preferably 60 vol %, even more preferably 55 vol %, particularly preferably 50 vol %, and most preferably 40 vol %.

The lower limit value of the α-olefin content in the reaction system in the solvent replacement step is not particularly limited, and from the viewpoint of economic efficiency such as an increase in production cost required for reducing the α-olefin content in the reaction system, the lower limit value may be usually 0.000001 wt. %, more preferably 0.00001 wt. %, still more preferably 0.0001 wt. %, particularly preferably 0.001 wt. %, and most preferably 0.003 wt. %.

The upper limit of the moisture content of the reaction solvent in the solvent replacement step is not particularly limited, and is usually preferably 10 mol ppm or less, more preferably 1 mol ppm or less, still more preferably 0.1 mol ppm or less, particularly preferably 0.01 mol ppm or less, and most preferably 0.005 mol ppm or less, from the viewpoint of more efficiently performing the drying treatment with a drying agent.

The lower limit of the moisture content of the reaction solvent is not particularly limited, and may be usually 0.000001 mol ppm or more, more preferably 0.00001 mol ppm or more, still more preferably 0.0001 mol ppm or more, particularly preferably 0.001 mol ppm or more, and most preferably 0.003 mol ppm or more, from the viewpoint of economic efficiency such as an increase in production cost required for reducing the moisture content in the reaction solvent in the solvent replacement step.

The above upper and lower limits may be freely combined. For example, the moisture content of the reaction solvent in the solvent replacement step is not particularly limited, and may be 0.000001 mol ppm or more and 10 mol ppm or less, more preferably 0.00001 mol ppm or more and 1 mol ppm or less, still more preferably 0.0001 mol ppm or more and 0.1 mol ppm or less, particularly preferably 0.001 mol ppm or more and 0.01 mol ppm or less, and most preferably 0.003 mol ppm or more and 0.005 mol ppm or less.

### Measurement Step

In the measurement step, the moisture content in the reaction system is measured after the deactivator addition step and before the α-olefin low polymerization reaction step.

When the moisture content measured in the measurement step is equal to or less than a predetermined value (5) described later, the α-olefin low polymerization reaction step is initiated. As a result, the formation of a polymeric by-product that causes process blockage may be effectively suppressed while the catalytic activity and the product selectivity may be maintained well.

### Predetermined Value (5)

The predetermined value (5) is an upper limit allowable value regarding the moisture content when the low polymerization reaction step is initiated. The predetermined value (5) is also an upper limit allowable value of the moisture content applied when the moisture content in the reaction system is reduced in the solvent drying step. In addition, the predetermined value (5) is a value within a range in which the moisture content in the reaction system during production of the α-olefin low polymer does not cause a practical problem.

The predetermined value (5) may be, for example, a value empirically determined using a production apparatus or the like to be used or a value theoretically derived using simulation or the like.

The predetermined value (5) is not particularly limited. From the viewpoint of maintaining the catalytic activity and the product selectivity well and suppressing the formation of a polymeric by-product that causes process blockage when the low polymerization reaction of an α-olefin is initiated, the predetermined value (1) is preferably 10 mol ppm, more preferably 5.0 mol ppm, still more preferably 3.0 mol ppm, particularly preferably 1.0 mol ppm, and most preferably 0.5 mol ppm with respect to the total amount of the gas in the reaction system.

The lower limit value of the moisture content in the reaction system in the measurement step is not particularly limited, and is usually preferably 0.0001 mol ppm, more preferably 0.001 mol ppm, still more preferably 0.01 mol ppm, particularly preferably 0.1 mol ppm, and most preferably 0.3 mol ppm, from the viewpoint of economic efficiency such as an increase in production cost required to reduce the moisture content in the reaction system.

The moisture content in the reaction system in the measurement step may be measured using a known moisture content analyzer. The moisture content analyzer may be an offline type or an online type provided at any position in the reaction system. For the moisture content analysis, for example, a moisture content analyzer of an electric capacity type, an electric resistance type, a microwave type, a near-infrared ray type, a neutron type, or the like may be used.

### Low Polymerization Reaction Step of α-Olefin

The method for producing an α-olefin low polymer of the present invention further includes a low polymerization reaction step of an α-olefin, in which the α-olefin low polymer is produced by supplying the catalyst, the solvent, and the raw material α-olefin into the reaction system after the deactivator addition step.

### Raw Material α-Olefin

The raw material α-olefin is not particularly limited as long as it is a compound having a double bond at a terminal carbon atom.

Examples of the raw material α-olefin include substituted or unsubstituted α-olefins having from 2 to 30 carbon atoms. Examples thereof include ethylene, propylene, 1-butene, 1-hexene, 1-octene, 3-methyl-1-butene, and 4-methyl-1-pentene. The α-olefin as a raw material is preferably ethylene, propylene, or 1-butene, more preferably ethylene or 1-butene, and particularly preferably ethylene.

In the case of ethylene, use of a known selective catalyst is preferred because 1-hexene as a trimer thereof is produced in high yield with high selectivity. In the case of using ethylene as a raw material, the raw material may contain an impurity component other than ethylene. Examples of the impurity component include methane, ethane, acetylene, and carbon dioxide.

The amount of each impurity component is preferably 5 mol % or less, more preferably 1 mol % or less, still more preferably 0.5 mol % or less, even more preferably 0.1 mol % or less, particularly preferably 0.05 mol % or less, and most preferably 0.01 mol % or less, with respect to ethylene of the raw material. Limiting the amount of the impurity component to 5 mol % or less may suppress the formation of a catalyst species different from the intended catalyst species, which otherwise results from the influence of the coordination of the impurity component to the catalyst or the like, thereby enabling control of the reaction.

### α-Olefin Low Polymer

The α-olefin low polymer is produced by subjecting a raw material α-olefin to a low polymerization reaction. The low polymerization reaction of an α-olefin refers to oligomerization of a raw material α-olefin. Therefore, the α-olefin low polymer means an oligomer in which several raw material α-olefins are bonded.

The reaction liquid after the low polymerization reaction may contain one α-olefin low polymer or may be a mixture of a plurality of α-olefin low polymers. The α-olefin low polymer is an oligomer in which 2 to 10, preferably 2 to 5 molecules of the α-olefin as a raw material are bonded.

In the case of using ethylene as a raw material, the α-olefin low polymer as a target product is preferably a substituted or unsubstituted linear or branched α-olefin having from 4 to 10 carbon atoms, and more preferably an unsubstituted linear α-olefin having from 4 to 10 carbon atoms. Non-limiting examples of the ethylene low polymer include 1-butene as a dimer of ethylene, 1-hexene as a trimer, 1-octene as a tetramer, and 1-decene as a pentamer. Among these, 1-butene, 1-hexene, and 1-octene are preferred, 1-hexene and 1-octene are more preferred, and 1-hexene is particularly preferred. When the target product is 1-hexene, the content ratio of 1-hexene in the product is preferably 90 wt. % or more.

### Reaction Solvent

The low polymerization reaction of the α-olefin is carried out in a reaction solvent.

The reaction solvent is not particularly limited, and a saturated hydrocarbon is preferably used. Examples thereof include chain saturated hydrocarbons having from 1 to 20 carbon atoms or alicyclic saturated hydrocarbons having from 1 to 20 carbon atoms, such as butane, pentane, 3-methylpentane, n-hexane, n-heptane, 2-methylhexane, octane, cyclohexane, methylcyclohexane, 2,2,4-trimethylpentane, and decalin. Further, an aromatic hydrocarbon such as benzene, toluene, xylene, ethylbenzene, mesitylene, or tetralin may be used as a reaction solvent.

One of the reaction solvents may be used alone, or two or more thereof may be used in combination.

Among these reaction solvents, chain saturated hydrocarbons and alicyclic saturated hydrocarbons having from 4 to 10 carbon atoms are preferred from the viewpoint of suppressing the formation or precipitation of by-product polymers such as polyethylene and further from the viewpoint of high catalytic activity. Non-limiting examples thereof preferably include n-heptane and cyclohexane, and more preferably n-heptane.

The amount of the reaction solvent used is not particularly limited. The amount of the reaction solvent used is usually from 0.5 to 5.0 times, preferably from 1.0 to 2.5 times, in terms of weight ratio, the amount of the raw material α-olefin supplied to the reactor. The supply amount of the raw material α-olefin as referred to herein is the sum of the consumption amount of the raw material α-olefin reacted in the reactor and the dissolution amount of the raw material α-olefin dissolved in the reaction solvent.

### Catalyst

The catalyst is not particularly limited as long as it is a catalyst capable of forming an α-olefin low polymer by subjecting an α-olefin to a low polymerization reaction. As the catalyst component, a catalyst system including a combination of a transition metal-containing compound (a) described later and an aluminum-containing compound (c) described later is preferably used. In addition to these two catalyst components, at least one of the halogen-containing compound (d) represented by general formula (I) described later or the nitrogen-containing compound (b) described later is more preferably used in combination.

### Transition Metal-Containing Compound (a)

The transition metal-containing compound (a) is suitably used as a constituent component of a catalyst.

The transition metal contained in the transition metal-containing compound (a) is not particularly limited, and transition metals of Groups 4 to 6 of the periodic table are preferably used. The transition metal contained in the transition metal-containing compound (a) is preferably one or more metals selected from the group consisting of chromium, titanium, zirconium, vanadium and hafnium, more preferably either or both of chromium and titanium, and most preferably chromium.

The transition metal-containing compound (a) used as a raw material of the catalyst includes one or more compounds represented by the general formula MeZn. In the general formula, Me represents a transition metal element, and Z represents an arbitrary organic group, inorganic group, or electronegative atom. n represents an integer of 1 to 6, and is preferably 2 or more. When n is 2 or more, Z may be the same or different from each other. The organic group may be a hydrocarbon group having from 1 to 30 carbon atoms optionally having a substituent. Non-limiting examples thereof include a carbonyl group, an alkoxy group, a carboxyl group, a β-diketonate group, a β-ketocarboxyl group, a β-ketoester group, and an amide group.

Non-limiting examples of the inorganic group Z include metal salt forming groups such as a nitric acid group and a sulfuric acid group.

Non-limiting examples of the electronegative atom Z include an oxygen atom and a halogen atom. However, the transition metal-containing compound (a) containing a halogen atom is not included in the halogen-containing compound (d) described later.

In the case of the transition metal-containing compound (a) in which the transition metal is chromium (hereinafter sometimes referred to as chromium-containing compound), non-limiting examples include chromium (IV)-tert-butoxide, chromium (III) acetylacetonate, chromium (III) trifluoroacetylacetonate, chromium (III) hexafluoroacetylacetonate, chromium (III) (2,2,6,6-tetramethyl-3,5-heptanedionate), Cr(PhCOCHCOPh)₃ (where Ph represents phenyl), chromium (II) acetate, chromium (III) acetate, chromium (III)₂-ethylhexanoate, chromium (III) benzoate, chromium (III) naphthenate, chromium (III) heptanoate, Cr(CH₃COCHCOOCH₃)₃, chromium (II) chloride, chromium (III) chloride, chromium (II) bromide, chromium (III) bromide, chromium (II) iodide, chromium (III) iodide, chromium (II) fluoride, and chromium (III) fluoride.

In the case of the transition metal-containing compound (a) in which the transition metal is titanium (hereinafter sometimes referred to as a titanium-containing compound), non-limiting example is TiCl₄, TiBr₄, TiI₄, TiBrCl₃, TiBr₂Cl₂, Ti(OC₂H₅)₄, Ti(OC₂H₅)₂Cl₂, Ti(O-n-C₃H₇)₄, Ti(O-n-C₃H₇)₂Cl₂, Ti(O-iso-C₃H₇)₄, Ti(O-iso-C₃H₇)₂Cl₂ Ti(O-n-C₄H₉)₄, Ti(O-n-C₄H₉)₂Cl₂, Ti(O-iso-C₄H₉)₄, Ti(O-iso-C₄H₉)₂Cl₂, Ti(O-tert-C₄H₉)₄, Ti(O-tert-C₄H₉)₂Cl₂TiCl₄(thf)₂ (where thf represents tetrahydrofuran), Ti((CH₃)₂N)₄, Ti((C₂H₅)₂N)₄, Ti((n-C₃H₇)₂N)₄, Ti((iso-C₃H₇)₂N)₄, Ti((n-C₄H₉)₂N)₄, Ti((tert-C₄H₉)₂N)₄, Ti(OSO₃CH₃)₄, Ti(OSO₃C₂H₅)₄, Ti(OSO₃C₃H₇)₄, Ti(OSO₃C₄H₉)₄, TiCp₂Cl₂, TiCp₂ClBr, Ti(OCOC₂H₅)₄, Ti(OCOC₂H₅)₂Cl₂, Ti(OCOC₃H₇)₄, Ti(OCOC₃H₇)₂Cl₂, Ti(OCOC₃H₇)₄, Ti(OCOC₃H₇)₂Cl₂, Ti(OCOC₄H₉)₄, Ti(OCOC₄H₉)₂Cl₂.

In the case of a transition metal-containing compound (a) in which the transition metal is zirconium (hereinafter sometimes referred to as a zirconium-containing compound), non-limiting examples include ZrCl₄, ZrBr₄, ZrI₄, ZrBrCl₃, ZrBr₂Cl₂, Zr(OC₂H₅)₄, Zr(OC₂H₅)₂Cl₂, Zr(O-n-C₃H₇)₄, Zr(O-n-C₃H₇)₂Cl₂, Zr(O-iso-C₃H₇)₄, Zr(O-iso-C₃H₇)₂Cl₂Zr(O-n-C₄H₉)₄, Zr(O-n-C₄H₉)₂Cl₂, Zr(O-iso-C₄H₉)₄, Zr(O-iso-C₄H₉)₂Cl₂, Zr(O-tert-C₄H₉)₄, Zr(O-tert-C₄H₉)₂Cl₂, Zr((CH₃)₂N)₄, Zr((C₂H₅)₂N)₄, Zr((n-C₃H₇)₂N)₄, Zr((iso-C₃H₇)₂N)₄, Zr((n-C₄H₉)₂N)₄, Zr((tert-C₄H₉)₂N)₄, Zr(OSO₃CH₃)₄, Zr(OSO₃C₂H₅)₄, Zr(OSO₃C₃H₇)₄, Zr(OSO₃C₄H₉)₄, ZrCp₂Cl₂, ZrCp₂ClBr, Zr(OCOC₂H₅)₄, Zr(OCOC₂H₅)₂Cl₂, Zr(OCOC₃H₇)₄, Zr(OCOC₃H₇)₂Cl₂, Zr(OCOC₃H₇)₄, Zr(OCOC₃H₇)₂Cl₂, Zr(OCOC₄H₉)₄, Zr(OCOC₄H₉)₂Cl₂, ZrCl₂ (HCOCFCOF)₂ZrCl₂ (CH₃COCFCOCH₃)₂.

In the case of a transition metal-containing compound (a) in which the transition metal is vanadium (hereinafter sometimes referred to as a vanadium-containing compound), non-limiting examples include vanadium pentoxide, vanadium oxytrichloride, vanadium oxytribromide, methoxyvanadate, ethoxyvanadate, n-propylvanadate, isopropoxyvanadate, n-butoxyvanadate, isobutoxyvanadate, t-butylvanadate, 1-methylbutoxyvanadate, 2-methylbutoxyvanadate, n-propoxyvanadate, neopentoxyvanadate, 2-ethylbutoxyvanadate, cyclohexylvanadate, allylcyclohexylvanadate, phenoxyvanadate, vanadium (III) acetylacetonate, vanadium (III) hexafluoroacetylacetonate, vanadium (III) (2,2,6,6-tetramethyl-3, 5-heptanedionate), V(C₆H₅COCHCOC₆H₅)₃, vanadium (III) acetate, vanadium (III)₂-ethylhexanoate, vanadium (III) benzoate, vanadium (III) naphthenate, V(CH₃COCHCOOCH₃)₃, vanadium (III) chloride, vanadium (III) bromide, vanadium (III) iodide, vanadium (III) fluoride, bis(cyclopentadienyl) vanadium dimethyl, bis(cyclopentadienyl) vanadium dimethyl chloride, bis(cyclopentadienyl) vanadium ethyl chloride, and bis(cyclopentadienyl) vanadium dichloride.

In the case of the transition metal-containing compound (a) in which the transition metal is hafnium (hereinafter sometimes referred to as the “hafnium-containing compound”), non-limiting examples include dimethylsilylene bis[1-(2-methyl-4-isopropyl-4H-azulenyl)]hafnium dichloride, dimethylsilylene bis[1-(2-methyl-4-phenyl-4H-azulenyl)]hafnium dichloride, dimethylsilylene bis[1-{2-methyl-4-(4-chlorophenyl)-4H-azulenyl}]hafnium dichloride, dimethylsilylene bis[1-{2-methyl-4-(4-fluorophenyl)-4H-azulenyl}]hafnium dichloride, dimethylsilylene bis[1-{2-methyl-4-(3-chlorophenyl)-4H-azulenyl}]hafnium dichloride, dimethylsilylene bis[1-{2-methyl-4-(2,6-dimethylphenyl)-4H-azulenyl}]hafnium dichloride, dimethylsilylene bis[1-(2-methyl-4,6-diisopropyl-4H-azulenyl)]hafnium dichloride, diphenylsilylene bis[1-(2-methyl-4-phenyl-4H-azulenyl)]hafnium dichloride, methylphenylsilylene bis[1-(2-methyl-4-phenyl-4H-azulenyl)]hafnium dichloride, methylphenylsilylene bis[1-{2-methyl-4-(1-naphthyl)-4H-azulenyl}]hafnium dichloride, dimethylsilylene bis[1-(2-ethyl-4-phenyl-4H-azulenyl)]hafnium dichloride, dimethylsilylene bis[1-{2-ethyl-4-(1-anthracenyl)-4H-azulenyl}]hafnium dichloride, dimethylsilylene bis[1-{2-ethyl-4-(2-anthracenyl)-4H-azulenyl}]hafnium dichloride, dimethylsilylene bis[1-{2-ethyl-4-(9-phenanthryl)-4H-azulenyl}]hafnium dichloride, dimethylmethylene bis[1-{2-methyl-4-(4-biphenylyl)-4H-azulenyl}]hafnium dichloride, dimethylgermylene bis[1-{2-methyl-4-(4-biphenylyl)-4H-azulenyl}]hafnium dichloride, dimethylsilylene bis[1-(2-ethyl-4-(3,5-dimethyl-4-trimethylsilylphenyl)-4H-azulenyl}]hafnium dichloride, dimethylsilylene 2[1-{2-methyl-4-(4-biphenylyl)-4H-azulenyl}][1-{2-methyl-4-(4-biphenylyl)indenyl}]hafnium dichloride, dimethylsilylene [1-(2-ethyl-4-phenyl-4H-azulenyl)][1-(2-methyl-4,5-benzoindenyl)]hafnium dichloride, dimethylsilylene bis[1-(2-methyl-4-phenylindenyl)]hafnium dichloride, dimethylsilylene bis[1-(2-methyl-4,5-benzoindenyl)]hafnium dichloride, and dimethylsilylene bis[1-{2-methyl-4-(1-naphthyl) indenyl}]hafnium dichloride.

One of the transition metal-containing compounds (a) may be used alone, or two or more thereof may be used in combination.

Among several transition metal-containing compounds (a), a chromium-containing compound is preferred, and among chromium-containing compounds, chromium (III)₂-ethylhexanoate is particularly preferred.

### Aluminum-Containing Compound (c)

The aluminum-containing compound (c) is also suitably used as a constituent component of the catalyst. As the aluminum-containing compound (c), the same compound as the alkylaluminum compound used in the solvent drying step described above may be used.

Examples of the aluminum-containing compound (c) include trialkylaluminum, alkylaluminum halide, alkoxyalkylaluminum, and alkylaluminum hydride.

Examples of the trialkylaluminum include trimethylaluminum, triethylaluminum, and triisobutylaluminum.

Examples of the alkylaluminum halide include diethylaluminum monochloride, ethylaluminum sesquichloride, and ethylaluminum dichloride.

Examples of the alkoxyalkylaluminum include diethylaluminum ethoxide.

Examples of the alkylaluminum hydride include diethylaluminum hydride.

One of the aluminum-containing compounds (c) may be used alone, or two or more thereof may be used in combination.

Among several aluminum-containing compounds (c), trialkylaluminum is preferred, and triethylaluminum is more preferred.

### Halogen-Containing Compound (d)

The catalyst may further contain a halogen-containing compound (d) as a constituent component thereof. As the halogen-containing compound (d), a compound represented by the following general formula (I) is used.

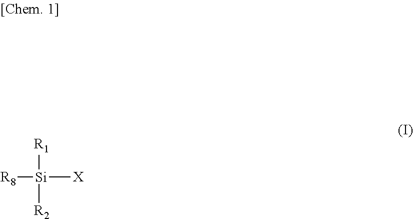

In formula (I), X represents a halogen atom. R₁ and R₂ are each independently a hydrogen atom, a hydrocarbon group optionally having a substituent, or a heteroatom-containing hydrocarbon group optionally having a substituent. R₈ is an aromatic group or an aromatic heterocyclic group optionally having a substituent.

X in formula (I) represents a halogen atom. Examples thereof include fluorine, chlorine, bromine, and iodine. X is preferably chlorine or bromine, and most preferably chlorine.

R₁ and R₂ in formula (I) may be the same or different from each other, and are each a hydrogen atom, a hydrocarbon group optionally having a substituent, or a heteroatom-containing hydrocarbon group optionally having a substituent. Examples of the hydrocarbon group include an alkyl group, a cycloalkyl group, a halogenated alkyl group, an allyl group, an aryl group, and a vinyl group. Examples of the heteroatom-containing hydrocarbon group include the oxygen-containing group, nitrogen-containing group, and sulfur-containing group of the hydrocarbon group exemplified above. R₁ and R₂ are preferably alkyl groups, and more preferably linear or branched alkyl groups having from 1 to 10 carbon atoms. When R₁ and R₂ are hydrocarbon groups, a substituent may be present on any carbon atom as long as the effect of the present invention is not significantly impaired, and examples of the substituent include an alkyl group, a cycloalkyl group, and an aryl group.

R₈ in formula (I) is an aromatic group or an aromatic heterocyclic group optionally having a substituent.

The aromatic group refers to a cyclic hydrocarbon group having aromaticity, and examples thereof include a phenyl group, an o-tolyl group, a m-tolyl group, a p-tolyl group, a 1-naphthyl group, a 2-naphthyl group, an o-xylyl group, a m-xylyl group, and a biphenylyl group.

The aromatic heterocyclic group is not particularly limited. Examples thereof include a furyl group, a thienyl group, a pyrrolyl group, an oxazolyl group, an isoxazolyl group, a thiazolyl group, an isothiazolyl group, an imidazolyl group, a pyrazolyl group, a pyridyl group, a pyrimidinyl group, a pyridazinyl group, a pyrazinyl group, a quinolyl group, and an isoquinolyl group.

Examples of the substituent which the aromatic group or the aromatic heterocyclic group may have include a halogen atom, an alkyl group, a cycloalkyl group, a halogenated alkyl group, an allyl group, an aryl group, a vinyl group, an oxygen-containing group, a nitrogen-containing group, and a sulfur-containing group.

The type of the halogen-containing compound represented by formula (I) is classified into an aromatic halogen-containing compound or a heteroaromatic halogen-containing compound.

Non-limiting example of the aromatic halogen-containing compound in which X in formula (I) is chlorine includes benzyl chloride, (1-chloroethyl)benzene, 2-methylbenzyl chloride, 3-methylbenzyl chloride, 4-methylbenzyl chloride, 4-ethylbenzyl chloride, 4-isopropylbenzyl chloride, 4-tert-butylbenzyl chloride, 4-vinylbenzyl chloride, α-ethyl-4-methylbenzyl chloride, α,α′-dichloro-o-xylene, α,α′-dichloro-m-xylene, α,α′-dichloro-p-xylene, 2,4-dimethylbenzyl chloride, 2,5-dimethylbenzyl chloride, 2,6-dimethylbenzyl chloride, 3,4-dimethylbenzyl chloride, 2,4,5-trimethylbenzyl chloride, 2,4,6-trimethylbenzyl chloride, 2,4,6-triisopropylbenzyl chloride, 2,3,5,6-tetramethylbenzyl chloride, 1-(chloromethyl) naphthalene, 1-(chloromethyl)-2-methylnaphthalene, 1,4-bis(chloromethyl)-2,3-dimethylnaphthalene, 1,8-bis(chloromethyl)-2,3,4,5,6,7-hexamethylnaphthalene, 9-(chloromethyl) anthracene, 9,10-bis(chloromethyl) anthracene, 7-(chloromethyl)benzanthracene, 7-chloromethyl-12-methylbenzanthracene, 2-chlorobenzyl chloride, 3-chlorobenzyl chloride, 4-chlorobenzyl chloride, 2-bromobenzyl chloride, 3-bromobenzyl chloride, 4-bromobenzyl chloride, 2-fluorobenzyl chloride, 3-fluorobenzyl chloride, 4-fluorobenzyl chloride, 2-nitrobenzyl chloride, 3-nitrobenzyl chloride, 4-nitrobenzyl chloride, 2-cyanobenzyl chloride, 3-cyanobenzyl chloride, 4-cyanobenzyl chloride, 2-methoxybenzyl chloride, 3-methoxybenzyl chloride, 4-methoxybenzyl chloride, 2-phenoxybenzyl chloride, 4-(methylthio)benzyl chloride, 4-(trifluoromethoxy)benzyl chloride, 1-(1-chloroethyl)-4-nitrobenzene, 2,3-dichlorobenzyl chloride, 2,4-dichlorobenzyl chloride, 2,6-dichlorobenzyl chloride, 3,4-dichlorobenzyl chloride, 2,4-difluorobenzyl chloride, 2,6-difluorobenzyl chloride, 2-chloro-4-fluorobenzyl chloride, 2-chloro-6-fluorobenzyl chloride, 4-bromo-2-fluorobenzyl chloride, 2-methyl-3-nitrobenzyl chloride, 4-methyl-3-nitrobenzyl chloride, 5-methyl-2-nitrobenzyl chloride, 2-methyl-2-phenoxybenzyl chloride, α,α′,2,3,5,6-hexachloro-p-xylene, and α,α′,2,4,5,6-hexachloro-m-xylene.

Non-limiting examples of the aromatic halogen-containing compound in which X in formula (I) is bromine include benzyl bromide, (1-bromoethyl)benzene, 4-butylbenzyl bromide, α-bromo-o-xylene, α-bromo-m-xylene, α-bromo-p-xylene, α,α′-dibromo-o-xylene, α,α′-dibromo-m-xylene, α,α′-dibromo-p-xylene, 3,5-di-tert-butylbenzyl bromide, 2-(bromomethyl) naphthalene, 1,4-bis(bromomethyl) naphthalene, 2-chlorobenzyl bromide, 3-chlorobenzyl bromide, 4-chlorobenzyl bromide, 2-bromobenzyl bromide, 3-bromobenzyl bromide, 4-bromobenzyl bromide, 2-fluorobenzyl bromide, 3-fluorobenzyl bromide, 4-fluorobenzyl bromide, 2-iodobenzyl bromide, 3-iodobenzyl bromide, 4-iodobenzyl bromide, 2-nitrobenzyl bromide, 3-nitrobenzyl bromide, 4-nitrobenzyl bromide, 2-cyanobenzyl bromide, 4-cyanobenzyl bromide, 2-(trifluoromethoxy)benzyl bromide, 4-(trifluoromethoxy)benzyl bromide, 4-(trifluoromethylthio)benzyl bromide, 2,3-dichlorobenzyl bromide, 3,4-dichlorobenzyl bromide, 2,5-dichlorobenzyl bromide, 2,6-dichlorobenzyl bromide, 2,4-difluorobenzyl bromide, 3,4-difluorobenzyl bromide, 2,5-difluorobenzyl bromide, 3,5-difluorobenzyl bromide, 2,6-difluorobenzyl bromide, 3,5-dimethoxybenzyl bromide, 3,5-dibenzyloxybenzyl bromide, 2-chloro-4-fluorobenzyl bromide, 2-chloro-5-fluorobenzyl bromide, 2-chloro-6-fluorobenzyl bromide, 3-chloro-2-fluorobenzyl bromide, 3-bromo-4-fluorobenzyl bromide, 4-bromo-2-fluorobenzyl bromide, 5-chloro-2-nitrobenzyl bromide, 2-hydroxy-5-nitrobenzyl bromide, 3-(2-fluoromethoxy)benzyl bromide, 3-(4-fluorophenoxy)benzyl bromide, 2,3,4-trifluorobenzyl bromide, 2,4,5-trifluorobenzyl bromide, 3,4,5-trifluorobenzyl bromide, 3,5-bis(3,5-dimethoxybenzyloxy)benzyl bromide, 3,5-bis(tert-butylthio)benzyl bromide, 3,5-bis[3,5-bis(3,5-benzyloxy)benzyloxy]benzyl bromide, pentafluorobenzyl bromide, 2,3,4,5-tetrafluorobenzyl bromide, and 2,3,5,6-tetrafluorobenzyl bromide.

Examples of the aromatic halogen-containing compound in which X in formula (I) is fluorine or iodine include benzyl fluoride and benzyl iodide.

Non-limiting examples of the heteroaromatic halogen-containing compound include 2,6-bis(chloromethyl)pyridine, 2,6-bis(bromomethyl)pyridine, 2-(chloromethyl)-5-nitrofuran, 2-chloro-5-(chloromethyl)pyridine, and 2-chloro-5-(chloromethyl)thiophene.

One of the halogen-containing compounds (d) may be used alone, or two or more thereof may be used in combination.

Among several halogen-containing compounds (d), an aromatic halogen-containing compound in which X in formula (I) is chlorine or bromine is preferred from the viewpoint of improving the catalytic activity and improving the selectivity of the target product. Among them, benzyl chloride is more preferred from the viewpoint of availability and little influence of a decomposition product on the reaction.

In addition to the halogen-containing compound (d), halogen-containing compounds such as 5-chloro-1,3-pentadiene, 5-methyl-1,2,3,4,5-pentachloro-1,3-cyclopentadiene, and 5-chloro-1,3-cyclopentadiene may also be used as catalyst components as necessary as long as the effects of the invention are not impaired.

### Nitrogen-Containing Compound (b)

The catalyst may further contain a nitrogen-containing compound (b) as a constituent component thereof in addition to the three catalyst components of the transition metal-containing compound (a), the aluminum-containing compound (c), and the halogen-containing compound. Examples of the nitrogen-containing compound include amines, amides, and imides.

Examples of the amine include pyrrole compounds. Non-limiting examples include pyrrole, 2,4-dimethylpyrrole, 2,5-dimethylpyrrole, 2-methyl-5-ethylpyrrole, 2,5-dimethyl-3-ethylpyrrole, 3,4-dimethylpyrrole, 3,4-dichloropyrrole, 2,3,4,5-tetrachloropyrrole, 2-acetylpyrrole, pyrroles such as dipyrrole in which two pyrrole rings are bonded via a substituent, and derivatives thereof. Examples of the derivative include metal pyrrolide derivatives. Examples thereof include aluminum pyrrolides such as diethylaluminum pyrrolides, ethylaluminum dipyrrolides, aluminum tripyrrolides, diethylaluminum (2,5-dimethylpyrrolides), ethylaluminum bis(2,5-dimethylpyrrolides), and aluminum tris(2,5-dimethylpyrrolides); sodium pyrrolides such as sodium pyrrolides and sodium (2,5-dimethylpyrrolides); lithium pyrrolides such as lithium pyrrolides and lithium (2,5-dimethylpyrrolides); and potassium pyrrolides such as potassium pyrrolides and potassium (2,5-dimethylpyrrolides). However, the above-mentioned aluminum-containing compound (c) does not contain aluminum pyrrolide. The halogen-containing compound does not contain the halogen-containing pyrrole compound.

One of the amines may be used alone or two or more thereof may be used in combination.

Examples of the amide include acetamide, N-methylhexanamide, succinamide, maleamide, N-methylbenzamide, imidazole-2-carboxamide, di-2 tenoylamine, β-lactam, δ-lactam, ε-caprolactam, and salts thereof with metals of Groups 1, 2, or 13 of the Periodic Table. One of the amides may be used alone or two or more thereof may be used in combination.

Examples of the imide include 1,2-cyclohexanedicarboximide, succinimide, phthalimide, maleimide, 2,4,6-piperidinetrione, perhydroazecine-2,10-dione, and salts thereof with metals of Group 1, 2, or 13 of the periodic table. Examples of the sulfonamide and sulfonimide include benzenesulfonamide, N-methylmethanesulfonamide, N-methyltrifluoromethylsulfonamide, and salts thereof with metals of Group 1, 2 or 13 of the periodic table.

One of the imides may be used alone or two or more thereof may be used in combination.

One of the nitrogen-containing compounds (b) may be used alone, or two or more thereof may be used in combination.

Among several nitrogen-containing compounds (b), an amine is preferred, the amine is more preferably a pyrrole compound, and the pyrrole compound is preferably 2,5-dimethylpyrrole and diethylaluminum (2,5-dimethylpyrrolide).

### Pre-Preparation of Catalyst

The catalyst used in the low polymerization reaction is preferably brought into contact with the raw material α-olefin in a state in which the transition metal-containing compound (a) and the aluminum-containing compound (c) are not in contact with each other in advance or in a state in which the contact time is short. By bringing into contact in such a state, a low polymerization reaction of the raw material α-olefin may be selectively performed, and a raw material α-olefin low polymer is produced in high yield.

The “state in which the transition metal-containing compound (a) and the aluminum-containing compound (c) are not in contact with each other in advance, or a state in which the contact time is short” is maintained not only at the start of the low-weight reaction but also when the raw material α-olefin and each catalyst component are additionally supplied to the reactor thereafter. However, the specific state described above is a preferred condition during catalyst preparation, and the influence after catalyst preparation is limited. For example, when the prepared catalyst is recovered from the reaction system and reused, the catalyst may be reused regardless of the above conditions.

The reason why the activity of the α-olefin low polymerization reaction decreases when the transition metal-containing compound (a) and the aluminum-containing compound (c) are brought into contact with each other in advance is not clear, but is presumed as follows.

It is considered that when the transition metal-containing compound (a) and the aluminum-containing compound (c) are brought into contact with each other, a ligand exchange reaction proceeds between a ligand coordinated to the transition metal-containing compound (a) and an alkyl group in the aluminum compound, leading to destabilization. As a result, the decomposition reduction reaction of the alkyl-transition metal-containing compound (a) preferentially proceeds, and as a result, metallation inappropriate for the low polymerization reaction of the α-olefin occurs, and the activity of the low polymerization reaction of the α-olefin decreases.

When the catalyst is prepared with the transition metal-containing compound (a), the nitrogen-containing compound (b), the aluminum-containing compound (c), and the halogen-containing compound (d), the contacting of the components is usually performed by the following methods.
A method for introducing a transition metal-containing compound (a) into a solution containing a nitrogen-containing compound (b), an aluminum-containing compound (c), and a halogen-containing compound (d).
A method for introducing an aluminum-containing compound (c) into a solution containing a transition metal-containing compound (a), a nitrogen-containing compound (b), and a halogen-containing compound (d).
A method for introducing a nitrogen-containing compound (b) and an aluminum-containing compound (c) into a solution containing a transition metal-containing compound (a) and a halogen-containing compound (d).
A method for introducing a transition metal-containing compound (a) and a nitrogen-containing compound (b) into a solution containing an aluminum-containing compound (c) and a halogen-containing compound (d).
A method for introducing an aluminum-containing compound (c) and a halogen-containing compound (d) into a solution containing a transition metal-containing compound (a) and a nitrogen-containing compound (b).
A method for introducing a transition metal-containing compound (a) and a halogen-containing compound (d) into a solution containing a nitrogen-containing compound (b) and an aluminum-containing compound (c).
A method for introducing a transition metal-containing compound (a), a nitrogen-containing compound (b), and a halogen-containing compound (d) into a solution containing an aluminum-containing compound (c).
A method for introducing a nitrogen-containing compound (b), an aluminum-containing compound (c), and a halogen-containing compound (d) into a solution containing a transition metal-containing compound (a).
A method for simultaneously and independently introducing a transition metal-containing compound (a), a nitrogen-containing compound (b), an aluminum-containing compound (c), and a halogen-containing compound (d) into a reaction system.

Each of the above solutions is usually prepared using the same solvent as the reaction solvent used in the low polymerization reaction.

### Supply Amount of Catalyst Component

The ratio of each constituent component of the transition metal atom-containing compound (a), the nitrogen atom-containing compound (b), and the alkylaluminum compound (c) is not particularly limited.

Usually, the nitrogen atom-containing compound (b) is from 1 mol to 100 mol, preferably from 2 mol to 50 mol, with respect to 1 mol of the transition metal atom of the transition metal atom-containing compound (a). The aluminum atom of the alkylaluminum compound (c) is from 1 mol to 2000 mol, preferably from 10 mol to 500 mol, with respect to 1 mol of the transition metal atom of the transition metal atom-containing compound (a).

When the halogen-containing compound (d) is used, the lower limit of the halogen-containing compound (d) is usually 1 mol, preferably 2 mol, still more preferably 3 mol, and the upper limit is usually 200 mol, preferably 150 mol, more preferably 100 mol, still more preferably 50 mol, with respect to 1 mol of the transition metal atom of the transition metal atom-containing compound (a). The number of moles of the transition metal atom-containing compound (a) with respect to 1 mole of transition metal atoms is synonymous with the molar multiple amount with respect to transition metal atoms in the low polymerization reaction system.

The amount of the catalyst composed of the transition metal-containing compound (a), the nitrogen-containing compound (b), the aluminum-containing compound (c), and the halogen-containing compound (d) to be used is not particularly limited, and is usually from 1.0×10⁻⁷ mol to 0.5 mol, preferably from 5.0×10⁻⁷ mol to 0.2 mol, and more preferably from 1.0×10⁻⁶ mol to 0.05 mol in terms of the transition metal element of the transition metal atom-containing compound (a) per liter of the reaction solvent described later.

### Low Polymerization Reaction Conditions

After it is confirmed that the moisture concentration in the reaction system falls within the above range, a catalyst and an α-olefin as a raw material are introduced into the reaction system to perform a low polymerization reaction of the α-olefin.

In the low polymerization reaction of the α-olefin, the ratio of each constituent component of the catalyst is usually from 1 mol to 50 mol, and preferably from 1 mol to 30 mol, with respect to 1 mol of the transition metal-containing compound (a). The amount of the aluminum-containing compound (c) is from 1 mol to 200 mol, preferably from 10 mol to 150 mol, with respect to 1 mol of the transition metal-containing compound (a). When the nitrogen-containing compound (b) is used, the amount of the nitrogen-containing compound (b) is from 1 mol to 50 mol, preferably from 1 mol to 30 mol, with respect to 1 mol of the transition metal-containing compound (a).

The amount of the catalyst used is not particularly limited, and is usually an amount such that the transition metal of the transition metal-containing compound (a) is from 1.0×10⁻⁹ mol to 0.5 mol, preferably from 5.0×10⁻⁹ mol to 0.2 mol, and more preferably from 1.0×10⁻⁸ mol to 0.05 mol, per liter of the solvent. Alternatively, the weight ratio of the amount of the catalyst used to the amount of the raw material α-olefin supplied to the reactor is usually from 1.0×10⁻⁸ to 1.0×10⁻³, preferably from 1.0×10⁻⁷ to 1.0×10⁻⁴. The supply amount of the raw material α-olefin as referred to herein is the sum of the consumption amount of the raw material α-olefin reacted in the reactor and the dissolution amount of the raw material α-olefin dissolved in the reaction solvent.

By using such a catalyst, for example, when ethylene is used as a raw material, hexene which is a trimer of ethylene is produced with a selectivity of 90% or more. Furthermore, in this case, the ratio of 1-hexene to hexene is 99% or more.

The reaction temperature of the low polymerization reaction of the α-olefin is not particularly limited, and is usually from 0 to 250° C., preferably from 50 to 200° C., and more preferably from 80 to 170° C.

The reaction pressure in the low polymerization reaction of the α-olefin is not particularly limited, and is usually in the range of normal pressure to 250 kgf/cm², preferably from 5 to 150 kgf/cm², and more preferably from 10 to 100 kgf/cm².

The residence time in the reactor is not particularly limited, and is usually in the range of 1 minute to 10 hours, preferably 3 minutes to 3 hours, and more preferably 5 minutes to 60 minutes.

The reaction form of the low polymerization reaction of the α-olefin is not particularly limited, and may be any of a batch type, a semi-batch type, or a continuous type.

### Moisture Content in Reaction System During Low Polymerization Reaction of α-Olefin

When the above-described α-olefin low polymerization reaction is performed, the moisture content with respect to the solvent in the reactor may be calculated by directly measuring the moisture concentration in the reactor in which the α-olefin low-polymer reaction is performed, or by measuring in advance the weight of moisture associated with the raw material, the solvent, the catalyst component, and the like supplied into the reactor, and dividing the total weight by the weight of the solvent supplied into the reactor.

From the viewpoint of simplicity of measurement and ease of management of measured values, it is preferable that the weight of moisture associated with the raw material, the solvent, the catalyst component, and the like supplied into the reactor is measured in advance, and the total weight thereof is divided by the weight of the solvent supplied into the reactor to calculate the moisture content (unit: mass ppm) with respect to the solvent in the reactor.

The moisture concentration in the reaction liquid may be accurately measured by the Karl Fischer titration. The moisture concentration in the raw material gas may be accurately measured by using a dew point meter.

The upper limit of the moisture content with respect to the solvent in the reactor when performing the low polymerization reaction of the α-olefin is not particularly limited. As the value of the moisture content increases, the polymer selectivity increases, thereby raising a risk of blockage in the process, and the catalytic activity also tends to decrease. Therefore, the upper limit may be usually preferably 5.0 mass ppm or less, more preferably 1.5 mass ppm or less, and still more preferably 0.5 mass ppm or less with respect to the total mass of the solvent in the reactor.

The lower limit of the moisture content is not particularly limited. Since the equipment cost and load at the time of removing moisture tend to increase as the value of the moisture content decreases, the lower limit may be usually set to preferably 0.0001 mass ppm or more, more preferably 0.001 mass ppm or more, and still more preferably 0.01 mass ppm or more with respect to the total mass of the solvent in the reactor.

The upper limit value and the lower limit value of the moisture content may be arbitrarily combined. When the low polymerization reaction of the α-olefin is performed, the moisture content with respect to the solvent in the reactor may be set to preferably 0.0001 mass ppm or more and 5.0 mass ppm or less, more preferably 0.001 mass ppm or more and 1.5 mass ppm or less, and still more preferably 0.01 mass ppm or more and 0.5 mass ppm or less, with respect to the total mass of the solvent in the reactor.

The method for reducing the moisture content of the reaction solvent is not particularly limited. For example, when n-heptane is used as a reaction solvent, normally commercially available n-heptane may contain moisture of about 20 to 30 mass ppm. Therefore, it is preferred to reduce the moisture content in the reaction solvent and the raw material α-olefin in advance using a drying agent such as molecular sieve before the reaction solvent and the raw material α-olefin are supplied to the reactor. If necessary, moisture remaining in the reaction solvent may be further removed by distillation separation.

### Method for Producing 1-Hexene

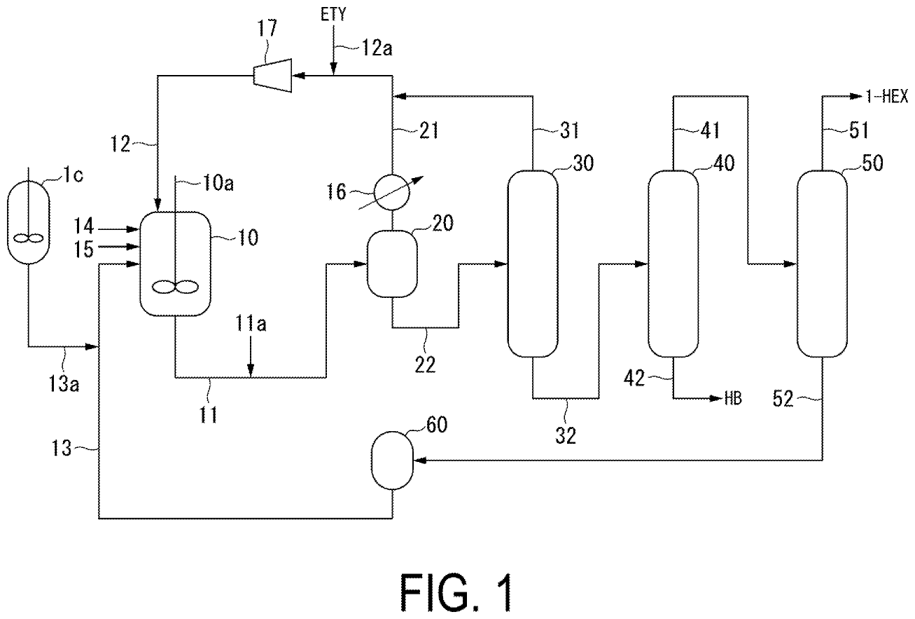

Next, a case where ethylene is used as the α-olefin and 1-hexene as a trimer thereof is produced as a low polymer will be described in more detail with reference to FIG. 1.

However, the dimensional ratios in the drawings are for convenience of description and are different from actual ones. In the following drawings, the same components are denoted by the same reference numerals, and redundant description may be omitted.

FIG. 1 illustrates an example of an apparatus for producing an α-olefin low polymer. The overall schematic diagram of the apparatus for producing 1-hexene using ethylene as a raw material illustrated in FIG. 1 shows a completely mixed stirring type reactor 10 for polymerizing ethylene in the presence of a catalyst, a degassing tank 20 for separating unreacted ethylene gas from the reaction liquid extracted from the reactor 10, an ethylene separation column 30 for distilling ethylene in the reaction liquid extracted from the degassing tank 20, a high-boiling separation column 40 for separating a high boiling point substance (hereinafter sometimes referred to as “HB: high boiler”) in the reaction liquid extracted from the ethylene separation column 30, and a hexene separation column 50 for distilling the reaction liquid extracted from the column top of the high-boiling separation column 40 and distilling 1-hexene (1-HEX). In addition, there is provided a compressor 17 that circulates unreacted ethylene separated in the degassing tank 20 and the condenser 16 to the reactor 10 via a circulation pipe 21.

In FIG. 1, examples of the reactor 10 include a known reactor equipped with a stirrer 10a, a baffle (not shown), and a jacket (not shown). As the stirrer 10a, a stirring blade of a type such as a paddle, a Faulder, a propeller, or a turbine is used in combination with a baffle such as a flat plate, a cylinder, or a hairpin coil as necessary.

### Gas Drying Step

In the gas drying step, when the production operation of the α-olefin low polymer is started, dry nitrogen gas is supplied into the reaction system from the ethylene supply pipe 12a in FIG. 1 and a nitrogen gas supply port (not shown) on the catalyst tank 1c. In the production apparatus shown in FIG. 1, the catalyst tank 1c, the reactor 10, the condenser 16, the compressor 17, the degassing tank 20, the ethylene separation column 30, the high-boiling separation column 40, the hexene separation column 50, the solvent drum 60, and the entire process piping (11, 11a, 12, 13, 13a, 21, 22, 31, 32, 41, 42, 51, 31, and 52) are collectively referred to as “in the reaction system”.

First, dry nitrogen gas having a moisture content of 2 mol ppm or less or a dew point of −70° C. or less is supplied into the reaction system. Supply of a gas such as dry nitrogen gas is continued until the moisture content measured at an arbitrary position (for example, the reactor 10, the degassing tank 20, the ethylene separation column 30, the high-boiling separation column 40, and the hexene separation column 50 in FIG. 1 may be used.) in the reaction system reaches a predetermined value (1) or less.

At this time, the temperature in the reaction system is not particularly controlled, and may be established in accordance with the outside air temperature.

The method for measuring the moisture content is as follows in the case where the sample is in a liquid state and in the case where the sample is in a gaseous state.

The moisture content of the liquid sample may be measured in accordance with ISO6358:1989, “Measuring instruments for industrial process measurement control—general use dew point measuring device”, using a coulometric titration type moisture meter (available from Mitsubishi Chemical Corporation, former company name; model: CA-06).

The moisture content of the gaseous sample may be determined by measuring the dew point using a dew point meter (available from TEKHNE Corporation, model: TK-100) in accordance with the dew point measurement method described in ISO 5725-2:1994 “Accuracy (trueness and precision) of measurement methods and results

Part 2: Basic method for the determination of repeatability and reproducibility of a standard measurement method”, and then calculating the moisture content (unit: mass ppm) from the dew point using the amount of water molecules (unit: mol) in the gas calculated using the relationship between the saturated vapor pressure and the temperature described in JISZ 8806, the amount of nitrogen molecules (unit: mol), and the following formula.

Moisture content (unit: mol ppm) in reactor (or process piping)=[number of moles of water molecules in reactor (or process piping)]/[number of moles of nitrogen molecules in reactor (or process piping)]

### Raw Material Replacement Step

Next, the dry nitrogen gas in the reaction system is replaced with the gas of the raw material α-olefin (for example, ethylene) supplied from the ethylene supply pipe 12a in FIG. 1.

At this time, the nitrogen gas may be replaced with the raw material olefin gas until the content of the nitrogen gas contained in the gas phase in the reaction system measured at an arbitrary position (the reactor 10 in FIG. 1, the high-boiling separation column 40, and the hexene separation column 50) in the reaction system becomes 20 vol % or less.

### Solvent Replacement Step

The above-described reaction solvent is supplied into the reaction system from the second supply pipe 13 in FIG. 1 using a known liquid feeding means such as a pump or pressure feeding. The reaction solvent after liquid passage may be discharged to the outside of the reaction system from an arbitrary position in the process, for example, the bottom of each distillation column (ethylene separation column 30, high-boiling separation column 40, and hexene separation column 50).

The supply amount of the reaction solvent in the solvent replacement step is not particularly limited as long as the reaction system is substantially filled with the reaction solvent, and may be usually 1.5 times or more the total volume to be subjected to solvent substitution as described above.

### Solvent Drying Step

In the solvent drying step, a drying agent-containing solvent is supplied into the reaction system. In the example shown in FIG. 1, a reaction solvent liquid containing an alkylaluminum compound functioning as a drying agent is supplied into the reaction system.

For example, a reaction solvent liquid (for example, a heptane solution having a TEA content of 10 mass %) containing an alkylaluminum compound (for example, triethyl aluminum (TEA)) may be supplied from the third supply pipe 14 in FIG. 1 into the reaction system using a known liquid feeding means such as a pump or pressure feeding. At this time, the reaction solvent liquid may be supplied until the moisture content in the process liquid in the reaction system becomes 1 mol ppm or less.

The moisture content in the reaction system may be measured by using an aquameter provided in the reactor 10 or the process piping (for example, pipe 11 connecting reactor 10 and degassing tank 20).

The reaction solvent liquid after liquid passage may be discharged to the outside of the reaction system from a discharge port provided at any position in the process, for example, the bottom of the reactor 10 or each distillation column (ethylene separation column 30, high-boiling separation column 40, and hexene separation column 50), pipes (11,22,32,41,42, and 51) connected to the reactor 10 or the distillation column, or the like.

### Deactivator Addition Step

The solvent drying step may include a deactivator addition step in which the above-described deactivator is supplied from a deactivator supply pipe 11a provided in the middle of the pipe 11 to the reaction liquid continuously extracted from the pipe 11 from the bottom of the reactor 10. The alkylaluminum compound in the reaction liquid is deactivated by the deactivator.

Furthermore, the reaction liquid after the deactivator is supplied is transferred to the degassing tank 20 through the pipe 11, and sequentially transferred to the ethylene separation column 30, the high-boiling separation column 40, and the hexene separation column 50.

The reaction liquid containing the deactivator after liquid passage may be discharged to the outside of the reaction system from a discharge port provided at an arbitrary position in the process, for example, the bottom of the reactor 10 or each distillation column (ethylene separation column 30, high-boiling separation column 40, and hexene separation column 50), pipes (11, 22, 32, 41, 42, and 51) connected to the reactor 10 or the distillation column, or the like.

### Measurement Step

The moisture content in the reaction system may be measured by using an aquameter provided in the reactor 10 or the process piping (for example, pipe 11 connecting reactor 10 and degassing tank 20).

The reaction solvent liquid after liquid passage may be discharged to the outside of the reaction system from a discharge port provided at any position in the process, for example, the bottom of the reactor 10 or each distillation column (ethylene separation column 30, high-boiling separation column 40, and hexene separation column 50), pipes (11,22,32,41,42, and 51) connected to the reactor 10 or the distillation column, or the like.

### Low Polymerization Reaction Step

Next, a low polymerization reaction step of producing 1-hexene by low polymerization of ethylene will be described.

In the low polymerization reaction step, the above-described catalyst, the above-described reaction solvent, and ethylene as a raw material are supplied into the reaction system. 1-Hexene, which is a trimer of ethylene, is produced by subjecting ethylene to a low polymerization reaction.

Ethylene (ETY) is continuously supplied from ethylene supply pipe 12a illustrated in FIG. 1 to reactor 10 via compressor 17 and first supply pipe 12. Here, for example, in a case where the compressor 17 is a two-stage compression system, the electricity charge may be reduced by connecting the circulation pipe 31 in the first stage and connecting the circulation pipe 21 in the second stage. From the second supply pipe 13, a solvent used for the low polymerization reaction of ethylene is supplied to the reactor 10.

In some examples, the transition metal-containing compound (a) and the nitrogen-containing compound (b) previously prepared in catalyst tank 1c are supplied from second supply pipe 13 to reactor 10 via catalyst supply pipe 13a. The aluminum-containing compound (c) is supplied from the third supply pipe 14 to the reactor 10. The halogen-containing compound (d) is supplied from the fourth supply pipe 15 to the reactor 10.

In some other examples, the halogen-containing compound (d) may be fed from the second supply line 13 to the reactor 10 via a feed tube (not shown). In addition, the aluminum-containing compound (c) may also be supplied to the reactor 10 from the second supply pipe 13 to the reactor 10 via the catalyst supply pipe 13a as long as the contact time with the transition metal-containing compound (a) is within several minutes. In this method, if a static mixer or the like is installed between the second supply pipe 13 and the reactor 10, a uniform mixed liquid of the catalyst components may be supplied to the reactor 10, so that the stirring power of the reactor 10 is reduced.

Furthermore, the trimerization reaction of ethylene is preferably performed so that the molar ratio of 1-hexene to ethylene in the reaction liquid ((molar concentration of 1-hexene in reaction liquid)/(molar concentration of ethylene in reaction liquid)) is from 0.05 to 1.5, particularly from 0.10 to 1.0.

In the case of a continuous reaction, it is preferred to adjust the catalyst concentration, the reaction pressure, and other conditions so that the molar ratio of ethylene and 1-hexene in the reaction liquid falls within the above range. In the case of a batch reaction, it is preferred to stop the trimerization reaction of ethylene when the molar ratio is within the above range.

By performing the trimerization reaction of ethylene under such conditions, by-production of a component having a boiling point higher than that of 1-hexene is suppressed, and the selectivity of 1-hexene tends to be further increased.

Next, the reaction liquid continuously extracted from the bottom of the reactor 10 through the pipe 11 is supplied to the degassing tank 20 after the trimerization reaction of ethylene is stopped by the deactivator supplied from the deactivator supply pipe 11a.

In the degassing tank 20, unreacted ethylene is degassed from the upper part. The degassed unreacted ethylene is circulated and supplied to the reactor 10 via the circulation pipe 21, the condenser 16, the compressor 17, and the first supply pipe 12. The reaction liquid from which unreacted ethylene has been removed is extracted from the bottom of the degassing tank 20.

The operating condition of the degassing tank 20 is usually a temperature of from 0° C. to 250° C., preferably from 50° C. to 200° C., and the pressure is normal pressure to 150 kgf/cm², preferably normal pressure to 90 kgf/cm².

Subsequently, the reaction liquid from which unreacted ethylene is degassed in the degassing tank 20 is extracted from the tank bottom of the degassing tank 20 and supplied to the ethylene separation column 30 by the pipe 22. In the ethylene separation column 30, ethylene is distilled from the column top by distillation. Distilled ethylene is circularly supplied to reactor 10 through circulation pipe 31 and first supply pipe 12. The reaction liquid from which ethylene has been removed is extracted from the column bottom of the ethylene separation column 30.

Under the operating conditions of the ethylene separation column 30, the column top pressure is usually from normal pressure to 30 kgf/cm², preferably from normal pressure to 20 kgf/cm². The reflux ratio (R/D) is usually from 0 to 500, preferably from 0.1 to 100.

Next, the reaction liquid from which ethylene has been removed in the ethylene separation column 30 is extracted from the column bottom of the ethylene separation column 30 and supplied to the high-boiling separation column 40 by the pipe 32. In the high-boiling separation column 40, a high boiling point component (HB: high boiler) is extracted from the bottom of the column. The distillate from which the high boiling point component is separated by the pipe 42 is extracted from the column top.

Under the operating conditions of the high-boiling separation column 40, the column top pressure is usually from 0.1 kgf/cm² to 10 kgf/cm², preferably from 0.5 kgf/cm² to 5 kgf/cm². The reflux ratio (R/D) is usually from 0 to 100, preferably from 0.1 to 20.

Subsequently, the reaction liquid extracted as a distillate from the column top part of the high-boiling separation column 40 is supplied to the hexene separation column 50 by the pipe 41. In the hexene separation column 50, 1-hexene by distillation is distilled from the column top by the pipe 51. Heptane is extracted from the column bottom of the hexene separation column 50. The heptane is stored in the solvent drum 60 through the solvent circulation pipe 52, and is further circulated and supplied to the reactor 10 as a reaction solvent through the second supply pipe 13.

Under the operating conditions of the hexene separation column 50, the column top pressure is usually from 0.1 kgf/cm² to 10 kgf/cm², preferably from 0.5 kgf/cm² to 5 kgf/cm². The reflux ratio (R/D) is usually from 0 to 100, preferably from 0.1 to 20.

### Experimental Results

Embodiments of the present invention will be described in more detail below with reference to experimental examples. However, the present invention is not limited to the following experimental examples.

A value from various production conditions and evaluation results in the following experimental examples has a meaning as a preferable value of an upper limit or a lower limit in any one of the embodiments of the present invention. A preferable range with respect to the present invention may be a range defined by a combination of the value of the upper limit or the lower limit described above and a value in the following experimental examples or a value between the examples.

Hereinafter, methods for measuring and evaluating physical property values are as follows.

### Catalytic Activity Value

The catalytic activity [g/g-Cr] was determined by sampling the column top liquid (“1-HEX” in FIG. 1) of the hexene separation column 50, and dividing the weight (unit: g) of the α-olefin low polymer (1-hexene), which is a reaction product produced after the low polymerization reaction of the α-olefin (ethylene), by the atomic weight (unit: g) of the transition metal (chromium atom) in the transition metal catalyst component (a) used for the low polymerization reaction.

### 1-Hexene Selectivity

With respect to the selectivity of 1-hexene as a target product, composition analysis was performed using a gas chromatograph measuring device (Model: GC-17AAF, available from Shimadzu Corporation) and internal standard gas chromatography for each of a circulating heptane solvent and a bottom liquid of an ethylene separation column (ethylene separation column 30 in FIG. 1), and the selectivity of 1-hexene produced in the reactor was calculated.

### Polyethylene Selectivity

The selectivity of polyethylene as a by-product was calculated from polyethylene produced by sampling a bottom liquid of an ethylene separation column (ethylene separation column 30 in FIG. 1), cooling the bottom liquid to room temperature, and then drying a filtration (filter diameter: 0.2 μm) residue.

### Experiment Example 1

In the production apparatus shown in FIG. 1, the other end of a solvent circulation pipe 52 connected to the column bottom of a hexene separation column 50 was connected to a second supply pipe 13, and was changed so as to circulate directly to a reactor 10 without passing through a solvent drum 60. In the production process thus changed, ethylene (ETY) was used as a raw material α-olefin, and 1-hexene (1-HEX) was produced by a continuous low polymerization reaction of ethylene according to the following procedure.

### Gas Drying Step

Dry nitrogen gas having a moisture content of 1.5 mol ppm or less was supplied to reactor 10 having a capacity of 0.1 m³, and circulated throughout the reaction system. Dry nitrogen gas was supplied to the entire reaction system and circulated until the moisture content in the gas phase of reactor 10 became 3.5 mol ppm or less and the moisture content in the gas phase in high-boiling separation column 40 and hexene separation column 50 and in the pipes (41, 42, 51, and 52) became 4.5 mol ppm or less. At this time, the temperature in the reaction system was not particularly controlled, and was assumed to be established in accordance with the outside air temperature. The moisture content of each part was measured using a dew point meter.

In the gas phase of the reactor 10 before the gas drying step was performed, the moisture content in the high-boiling separation column 40, the hexene separation column 50, and each pipe (41, 42, 51, or 52) was 9300 mol ppm.

The dry nitrogen gas after ventilation was discharged to the outside of the reaction system from, for example, extraction ports (not shown) provided in the reactor 10, the degassing tank 20, the ethylene separation column 30, and the high-boiling separation column 40 shown in FIG. 1, the pipe 11, the first supply pipe 12, the circulation pipe 21, the pipe 22, the circulation pipe 31, the pipe 32, the pipe 41, the pipes 42 and 50, the pipe 51, and the solvent circulation pipe 52.

### Raw Material Replacement Step

Ethylene gas having a moisture content of 5 mol ppm or less was supplied to reactor 10, and circulated throughout the reaction system. Ethylene gas was supplied to the entire reaction system until the nitrogen gas content in the gas phase of the reactor 10 became 0.5 vol % or less, and the nitrogen gas content in the degassing phase 20, in the gas phase in the ethylene separation column 30, in each pipe (21, 22, 31), and as necessary, in the high-boiling separation column 40, in the gas phase in the hexene separation column 50, and in each pipe (41, 42, 51, or 52) became 0.5 vol % or less. At this time, the temperature in the reaction system was not particularly controlled, and was assumed to be established in accordance with the outside air temperature. The nitrogen gas content of each part was measured using a gas chromatograph in accordance with ASTM D2504.

The ethylene gas after ventilation was discharged to the outside of the reaction system through an extraction port (not shown) provided in the reactor 10 and an extraction port (not shown) provided in the circulation pipe 21 or the circulation pipe 31.

### Solvent Replacement Step

Heptane was supplied to the reactor 10 by using a pump, and passed through the entire reaction system to be sealed. The supply of heptane in this step was performed until the ethylene gas in the entire reaction system reached 60 vol % or less and the ethylene gas in the reactor reached 50 vol % or less. At this time, the temperature in the reaction system was not particularly controlled, and was assumed to be established in accordance with the outside air temperature.

Excess heptane after liquid passage was discharged to the outside of the reaction system from the bottom of the high-boiling separation column 40 shown in FIG. 1.

### Solvent Drying Step, Deactivator Addition Step, and Measurement Step

A TEA/heptane solution with a triethylaluminum (TEA) content of 10 mass % was fed into reactor 10 using a pump and circulated throughout the reaction system. In parallel with this operation, 2-ethylhexanol (2EH) alone as a deactivator for deactivating triethylaluminum was supplied to the pipe 11 through the deactivator supply pipe 11a.

The TEA/heptane solution was supplied to the entire reaction system until the moisture content of the liquid phase (TEA/heptane solution) in the reactor 10 measured using an online moisture content analyzer (not illustrated) provided on the first supply pipe 12 became 1 mol ppm or less, and the moisture content of the TEA/heptane solution in the first supply pipe 12 became 1 mol ppm or less. At this time, the temperature in the reaction system was not particularly controlled, and was assumed to be established in accordance with the outside air temperature. The TEA/heptane solution after liquid passage was discharged from the pipe 42 to the outside of the reaction system.

The moisture content in the reactor 10 and the process piping before starting the solvent drying step (moisture content before starting the solvent drying step) was 6 mol ppm. In the solvent drying step, the time required from the start of the operation until the moisture content of the TEA/heptane solution in the liquid phase of reactor 10 reached 1 mol ppm was 2880 minutes.

### Low Polymerization Reaction Step

According to the following procedure, n-heptane as a reaction solvent and ethylene as a raw material α-olefin were supplied to reactor 10, and then a catalyst component described later was supplied to perform a low polymerization reaction of ethylene to polymerize 1-hexene as an α-olefin low polymer.

As the catalyst component, chromium 2-ethylhexanoate was used as the chromium compound (a), dimethylpyrrole was used as the pyrrole compound (b), triethylaluminum diluted with n-heptane was used as the aluminum-containing compound (c), and hexachloroethane diluted with n-heptane was used as the halogen-containing compound (d) of the catalyst component.

The temperature inside the reactor 10 was raised to 140° C. to initiate a low polymerization reaction of ethylene. During the low polymerization reaction, the temperature inside the reactor 10 was maintained at 140° C., and the total pressure was maintained at 7 MPaG.

The reaction liquid was sampled 9 hours after the start of the low polymerization reaction, and the value of catalytic activity was 842000 g/g-Cr, the 1-hexene selectivity was 95.36 mass %, and the polyethylene (PE) selectivity was 0.08 mass, which were measured using the evaluation method described above. This result was at a level that was sufficiently satisfactory from an industrial point of view.

All the n-heptane used in Experimental Example 1 was previously dehydrated by molecular sieve. The moisture concentration before being supplied to the reaction system was measured and found to be 0.6 mass ppm. A coulometric titration type moisture meter (available from Mitsubishi Chemical Corporation, former company name; model: CA-06) was used for the measurement.

The moisture concentration in ethylene used in the reaction was 1.0 mass ppm as determined by dew point measurement. A dew point meter (available from TEKHNE Corporation, model: TK-100) was used for the measurement.

### Reference Experimental Example 1

In the present Reference Experimental Example 1, it is shown that as the moisture content in the reaction system increases, the catalytic activity tends to be impaired at the time of low polymerization of the α-olefin, and the formation of polyethylene as a by-product that causes process blockage increases.

Specifically, 1-hexene was produced using ethylene under conditions in which the moisture content in the reaction system was changed in the following procedure. Details will be described below.

### Reference Experimental Example 1-1

All the n-heptane used in this example was dehydrated in advance by molecular sieve, and the moisture concentration before being supplied to the reaction system was measured, and found to be 0.6 mass ppm. A coulometric titration type moisture meter (available from Mitsubishi Chemical Corporation, former company name; model: CA-06) was used for the measurement. The moisture concentration in ethylene used in the reaction was 1.0 mass ppm as determined by dew point measurement. A dew point meter (available from TEKHNE Corporation, model: TK-100) was used for the measurement.

First, under a nitrogen atmosphere, 0.26 g (2.73 mmol) of 2,5-dimethylpyrrole as the nitrogen-containing compound (b) as a catalyst component and 239 ml of n-heptane were charged into a 500 ml three-necked glass flask having a stirrer and previously heated and dried at 140° C. for 2 hours or more. Subsequently, as the aluminum-containing compound (c) as the catalyst component, 6.22 ml (2.73 mmol) of triethylaluminum diluted to 50 g/L with n-heptane was added. Thereafter, the temperature of the flask was raised, and n-heptane was refluxed at 98° C. for 3 hours under a nitrogen atmosphere to prepare aluminum pyrrolide as a nitrogen-containing compound. The resultant was then cooled to 80° C. Subsequently, as the transition metal-containing compound (a) of the catalyst component, 4.38 ml (0.45 mmol) of chromium 2-ethylhexanoate diluted to 50 g/L with n-heptane was added. After the addition, the mixture was heated and stirred at 80° C. for 30 minutes under a nitrogen atmosphere to prepare a catalyst liquid.

Next, a set of 500 ml autoclaves was heated and dried at 140° C. for 2 hours or more using a heating dryer, and immediately after being taken out from the dryer, components such as a catalyst feed pipe, a stirring unit, various valves, and various connection pipes were assembled. Next, the inside of the autoclave was purged with vacuum nitrogen. A catalyst feed pipe having a pressure-resistant rupture plate was attached to the autoclave. The catalyst feed pipe was charged with 2 mL of the above-described catalyst liquid. The autoclave vessel was charged with 162 mL of n-heptane as the reaction solvent, 5 mL of n-undecane as an internal standard for compositional analysis by gas chromatography, 3.36 mL (0.20 mmol) of a triethylaluminum solution diluted to 6.67 g/L with n-heptane as the aluminum-containing catalyst component (c), and 2 mL (0.044 mmol) of a benzyl chloride solution diluted to 2.76 g/L with n-heptane as the halogen-containing compound (d) as a catalyst component.

It was confirmed by analysis that water was not contained in the autoclave. The molar ratio of the moisture content to the Cr amount of chromium 2-ethylhexanoate (referred to as “H₂O/Cr molar ratio”) was “0”.

Next, the autoclave was heated to 140° C., and then ethylene was introduced from a catalyst feed pipe to start a low polymerization reaction of ethylene. During the reaction, the temperature inside the autoclave was maintained at 140° C. and the total pressure was maintained at 7 MPaG.

After 30 minutes, ethanol was added to stop the reaction. The total weight of ethylene introduced into the autoclave from the start of the reaction to the end of the reaction was 123 g, and the weight of n-heptane in the autoclave was 119 g. Then, the reaction liquid and the reaction gas were sampled, and the compositions of the whole hydrocarbon compound having 6 carbon atoms and 1-hexene and C10 to C30 in the hydrocarbon compound were analyzed by gas chromatography. The reaction liquid was filtered and dried, and then the concentration of the polymer solid contained in the reaction liquid was measured. The catalytic activity was determined by dividing the mass of the reaction product produced by the reaction for 30 minutes by the transition metal atom amount (grams) in the transition metal catalyst component used in the reaction.

During the low polymerization reaction step, the moisture content with respect to the solvent in the autoclave was calculated as follows. The total ethylene weight includes not only the ethylene weight consumed by the reaction, but also the unreacted ethylene weight remaining in the autoclave without reaction.

Moisture content relative to solvent in autoclave [mass ppm]=(weight of moisture in total ethylene [mg]+weight of moisture in solvent [mg])/weight of solvent [g]

The total weight of ethylene introduced into the autoclave in Experimental Example 1 was 123 g, and the moisture concentration contained in the used ethylene was 1.0 mass ppm. Thus, the weight of water contained in the total ethylene is 0.12 mg. Since the weight of n-heptane as the solvent is 119 g (175 ml) and the concentration of moisture contained therein is 0.6 mass ppm, the weight of water contained in the solvent is 0.07 mg. From the above, the moisture content relative to the solvent in the autoclave is 1.6 mass ppm.

In Experimental Example 1, the value of catalytic activity was 338843 g/g-Cr, the selectivity of a hydrocarbon compound having 6 carbon atoms was 96.9 mass %, the selectivity of 1-hexene in the hydrocarbon compound was 99.5 mass %, and the selectivity of polyethylene (PE) was 0.069 mass %, and these results were at an industrially sufficient level.

### Reference Experimental Examples 1-2, 1-3, and 1-4

Polymerization reaction of 1-hexene was performed under the same conditions as in Reference Experimental Example 1-1, except that water was added so that the molar ratio of the moisture content to the Cr amount was 9, 20, and 41, respectively. The evaluation results of the catalytic activity and the polyethylene (PE) selectivity are shown in Table 1.

TABLE 1

| | H₂O/Cr | Catalytic | |
|---|---|---|---|
| | molar | activity | PE |
| | ratio | value | selectivity |
| | — | [g/g-Cr] | % |
| Reference Experimental Example 1-1 | 0 | 338843 | 0.069 |
| Reference Experimental Example 1-2 | 9 | 326975 | 0.053 |
| Reference Experimental Example 1-3 | 20 | 287180 | 0.072 |
| Reference Experimental Example 1-4 | 41 | 136466 | 0.205 |

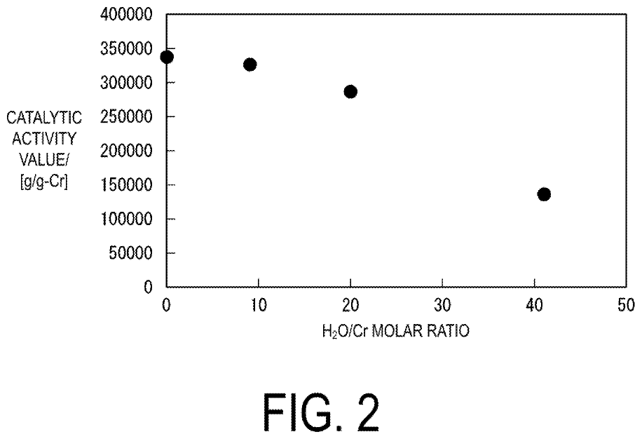

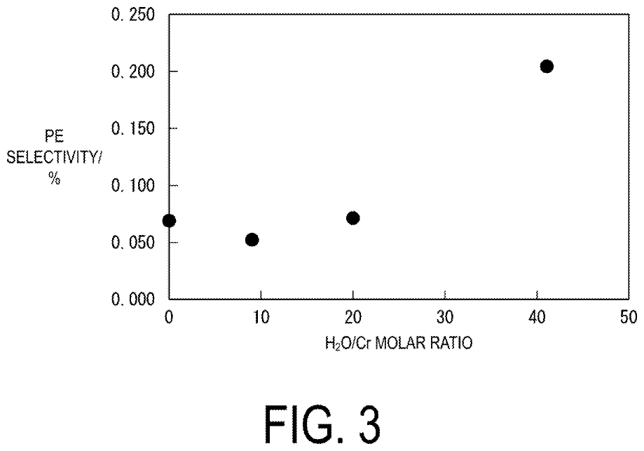

The evaluation results of the catalytic activity and the polyethylene selectivity of Reference Experimental Examples 1-1 to 1-4 are shown in the graph of FIG. 2 and the graph of FIG. 3, respectively.

As shown in Table 1, FIG. 2, and FIG. 3, it may be seen that as the moisture content in the reaction system increases, the catalytic activity tends to be impaired during low polymerization of the α-olefin, and the formation of a polymeric by-product that causes process blockage increases.

### Reference Experimental Example 2

In the present reference experimental example 2, it is shown that as the moisture content in the reaction system increases, the catalytic activity and the 1-hexene selectivity as a target product tend to be impaired during the low polymerization of the α-olefin, and the formation of polyethylene as a byproduct that causes process blockage increases.

Specifically, 1-hexene was produced using ethylene under conditions in which the moisture content in the reaction system was changed in the following procedure. Details will be described below.

### Reference Experimental Example 2-1

Chromium 2-ethylhexanoate as the transition metal-containing compound (a), 2,5-dimethylpyrrole as the nitrogen-containing compound (b), triethylaluminum as the aluminum-containing compound (c), hexachloroethane as the halogen-containing compound (d), and heptane as the reaction solvent were charged into an autoclave type reactor (capacity: 500 mL) as components of a catalyst. At that time, the H₂O/Cr molar ratio, the molar ratio of triethylaluminum to the Cr amount of chromium 2-ethylhexanoate (referred to as “TEA/Cr molar ratio”), the molar ratio of chlorine atoms of hexachloroethane to the Cr amount of chromium 2-ethylhexanoate (referred to as “Cl/Cr molar ratio”), and the total concentration of the four components of the catalyst (referred to as “catalyst concentration” in Table 2) were set to values shown in Table 2. Subsequently, ethylene was introduced as a raw material α-olefin in the same manner as in Reference Experimental Example 1 to start a low polymerization reaction of ethylene. During the reaction, the temperature inside the autoclave was maintained at 140° C. and the total pressure was maintained at 7 MPaG. It was confirmed by analysis that water was not contained in the reactor. Therefore, the molar ratio of the moisture content to the Cr amount of chromium 2-ethylhexanoate was “0”.

Subsequently, a polymerization reaction of 1-hexene was carried out under a pressure of 7 MPaG at a temperature of 140° C. for a reaction time of 60 minutes. The values of the catalytic activity, and the evaluation results of the selectivity of 1-hexene as a target product, the selectivity of the olefinic compound having from 4 to 22 carbon atoms excluding 1-hexene as a by-product, and the polyethylene selectivity are shown in Table 2.

### Reference Experimental Examples 2-2, 2-3, and 2-4

Water was further added from the conditions of Reference Experimental Example 1-1. A polymerization reaction of 1-hexene was performed under the same conditions as in Reference Experimental Example 2-1 except that the H₂O/Cr molar ratio, the TEA/Cr molar ratio, the Cl/Cr molar ratio, the catalyst concentration, and the moisture content were changed as shown in Table 2. The values of the catalytic activity, and the evaluation results of the selectivity of 1-hexene as a target product, the selectivity of the olefinic compound having from 4 to 22 carbon atoms excluding 1-hexene as a by-product, and the polyethylene selectivity are shown in Table 2.

TABLE 2

| | | Experimental | Experimental | Experimental | Experimental |
|---|---|---|---|---|---|
| | Unit | Example 2-1 | Example 2-2 | Example 2-3 | Example 2-4 |
| H₂O/Cr molar ratio | — | 0 | 80 | 240 | 240 |
| TEA/Cr molar ratio | — | 60 | 140 | 300 | 265 |
| Cl/Cr molar ratio | — | 6 | 6 | 6 | 6 |
| Moisture content in reaction | mass | 0 | 44 | 132 | 132 |
| system | ppm | | | | |
| Catalyst concentration | mg/L | 10 | 10 | 10 | 10 |
| Catalytic activity | g/g-Cr | 262277 | 255949 | 289231 | 310599 |
| Selectivity of compound having | mass % | 94.7 | 89.2 | 86.0 | 86.6 |
| 6 carbon atoms | | | | | |
| 1-Hexene selectivity in compound | mass % | 99.1 | 97.5 | 96.8 | 97.3 |
| having 6 carbon atoms | | | | | |
| Selectivity of olefinic compound | mass % | 5.5 | 11.1 | 14.6 | 13.7 |
| having from 4 to 22 carbon | | | | | |
| atoms, excluding 1-hexene | | | | | |
| Polyethylene selectivity | mass % | 0.08 | 0.18 | 0.29 | 0.85 |

As shown in Table 2, it is found that as the moisture content in the reaction system increases, the catalytic activity and the selectivity of 1-hexene as a target product tend to be impaired at the time of low polymerization of the α-olefin, and the selectivity of the olefin-based compound having from 4 to 22 carbon atoms excluding 1-hexene as a by-product and the selectivity of polyethylene that causes process blockage increase.

### Reference Experimental Example 3

This Reference Experimental Example 3 shows that by performing the gas drying step in the reaction system in advance using dry nitrogen before performing the solvent drying step using an alkylaluminum compound, the time required for the solvent drying step is shortened and the amount of the alkylaluminum compound used is reduced.

Specifically, 1-hexene was produced using ethylene while changing the moisture content in the reaction system in the following procedure. Details will be described below.

### Reference Experimental Example 3-1

A three-neck glass round bottom flask equipped with a three-way cock, a separatory funnel and a septum cap was charged with air having a moisture content of 1.0 mass % at normal temperature (25° C.) and atmospheric pressure. Then, a 10 mass % heptane solution of triethylaluminum was added from the separatory funnel into the round bottom flask, and the round bottom flask was sealed. Immediately after the addition of the heptane solution, the gas in the gas phase of the round bottom flask was collected from the septum cap portion over time using a microsyringe, and the moisture content contained in the gas was measured over time using a dew point meter.

### Reference Experimental Example 3-2

In Reference Experimental Example 3-1, air having a moisture content of 1.0 mass % was sealed in a round-bottom flask, and then several kinds of nitrogen gas having different moisture contents within a range of 1 mol ppm to 100 mol ppm were supplied into the round-bottom flask to completely replace the gas in the flask. Subsequently, in the same manner as in Reference Experimental Example 3-1, the moisture content in the gas phase of the round-bottom flask after adding a 10 mass % heptane solution of triethylaluminum was measured over time.

As in Reference Experimental Example 3-2, it is expected that the time required for the drying step using the alkylaluminum compound is shortened by performing the gas drying treatment using dry nitrogen in advance before drying the inside of the reaction system using the alkylaluminum compound. It is also expected that the amount of the alkylaluminum compound used is reduced.

### Reference Experimental Example 4

When the alkylaluminum compound supplied as a drying agent in the solvent drying step remains in the reaction system downstream of the reactor, the low polymerization reaction of the α-olefin continues at the start of the low polymerization reaction step, and by-products may be formed to decrease the product selectivity.

This Reference Experimental Example 4 shows that a decrease in the product selectivity due to formation of a by-product by the above-described alkylaluminum compound is more effectively suppressed by performing the deactivator addition step.

Specifically, a 1-hexene/heptane solution containing a catalyst and a drying agent (TEA) was subjected to heat reflux treatment by changing the type of alcohol added as a deactivator and the molar ratio of the deactivator to the drying agent (TEA) according to the following procedure, and the isomerization rate of 1-hexene was measured as a by-product selectivity. Details will be described below.

### Reference Comparative Experimental Example 4-1

In a round-bottom flask (volume: 100 mL), 20 mL of a 1-hexene/heptane solution (volume ratio: 1/5) was charged, and chromium 2-ethylhexanoate as the transition metal-containing compound (a), 2,5-dimethylpyrrole as the nitrogen-containing compound (b), triethylaluminum (TEA) as the aluminum-containing compound (c), and hexachloroethane as the halogen-containing compound (d) were further charged as the components of the catalyst. In this Experimental Example, TEA corresponds to the drying agent. The molar ratio of Cr in chromium 2-ethylhexanoate/2,5-dimethylpyrrole/hexachloroethane/aluminum in TEA was 1/6/6/600 (molar ratio).

Next, a reflux treatment was performed for 4 hours while maintaining the temperature in the round-bottom flask at 90° C. Before the reflux treatment, it was confirmed by analysis that water was not contained in the reactor.

After the reflux treatment, the 1-hexene/heptane solution in the round-bottom flask was collected, and the amount (unit: mol) of each by-product of trans-3-hexene, cis-3-hexene, trans-2-hexene, and cis-2-hexene formed, a by-product of 1-hexene, was measured with respect to 100% of the total number of moles of 1-hexene before the reflux treatment by an internal standard method using gas chromatography, and the result was taken as the selectivity (unit: mol %) of each by-product. Furthermore, the total value of the selectivities of these four by-products was taken as the isomerization rate (unit: mol %) of 1-hexene. The evaluation results are shown in Table 3.

### Reference Experimental Examples 4-2, 4-3, and 4-4

In Reference Comparative Experimental Example 4-1, 2-ethylhexanol (2EH) was added as a deactivator for TEA into the round-bottom flask before the reflux treatment was started. Reflux treatment was performed under the same conditions as in Reference Comparative Experimental Example 4-1 except that the molar ratio of the deactivator (2EH) to the drying agent (TEA) (referred to as “molar ratio of deactivator/drying agent (TEA)”) was added so as to have the value shown in Table 3.

The results of evaluation in the same manner as in Reference Comparative Experimental Example 4-1 are shown in Table 3.

### Reference Experimental Example 4-5

The reflux treatment was performed under the same conditions as in Reference Experimental Example 4-3, except that 1-butanol (1-BuOH) was used as the deactivator for TEA instead of 2-ethylhexanol (2EH) in Reference Experimental Example 4-3. The evaluation results are shown in Table 3.

### Reference Experimental Example 4-6

The reflux treatment was performed under the same conditions as in Reference Experimental Example 4-3, except that 2-propylheptanol (2PH) was used as the deactivator for TEA instead of 2-ethylhexanol (2EH) in Reference Experimental Example 4-3. The evaluation results are shown in Table 3.

TABLE 3

| | | Molar ratio of | | Selectivity | Selectivity | Selectivity | Isomerization |
|---|---|---|---|---|---|---|---|
| | | deactivator/drying | Selectivity of | of cis-3- | of trans-2- | of cis-2- | rate of 1- |
| | | agent (TEA) | trans-3-hexene | hexene | hexene | hexene | hexene |
| | Deactivator | (mol/mol) | (mol %) | (mol %) | (mol %) | (mol %) | (mol %) |
| Reference | Absent | 0.0 | 0.103 | 0.000 | 0.947 | 0.338 | 1.39 |
| Comparative | | | | | | | |
| Experimental | | | | | | | |
| Example 4-1 | | | | | | | |
| Reference | 2EH | 1.2 | 0.016 | 0.000 | 0.184 | 0.071 | 0.27 |
| Experimental | | | | | | | |
| Example 4-2 | | | | | | | |
| Reference | 2EH | 3.3 | 0.005 | 0.000 | 0.144 | 0.017 | 0.17 |
| Experimental | | | | | | | |
| Example 4-3 | | | | | | | |
| Reference | 2EH | 9.8 | 0.002 | 0.000 | 0.142 | 0.022 | 0.17 |
| Experimental | | | | | | | |
| Example 4-4 | | | | | | | |
| Reference | 1-BuOH | 3.3 | 0.005 | 0.000 | 0.125 | 0.022 | 0.15 |
| Experimental | | | | | | | |
| Example 4-5 | | | | | | | |
| Reference | | | | | | | |
| Experimental | 2PH | 3.3 | 0.005 | 0.000 | 0.175 | 0.031 | 0.21 |
| Example 4-6 | | | | | | | |

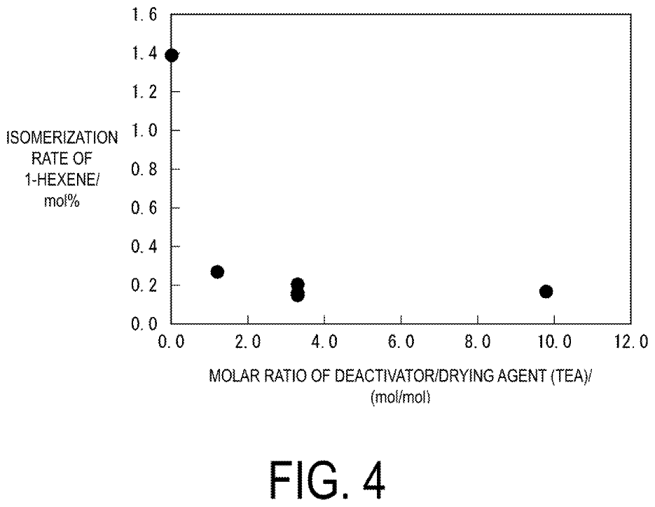

The evaluation results of the isomerization rate of 1-hexene for each of Experimental Examples 4-1 to 4-6 are shown in the graph of FIG. 4.

As shown in Table 3 and FIG. 4, when the alkylaluminum compound (TEA) remained in the reaction system as a drying agent, it was confirmed that the isomerization rate of the product (1-hexene) caused by the formation of a by-product could be effectively suppressed as the addition amount of the deactivator increased.

### INDUSTRIAL APPLICABILITY

According to the present invention, in producing an α-olefin low polymer by a low polymerization reaction of an α-olefin, the moisture content in the reaction system is efficiently reduced prior to the start of the production operation while suppressing the amount of an expensive alkylaluminum compound used as a drying agent, thus enabling the production operation to be started promptly. Furthermore, during the low polymerization of an α-olefin, the catalytic activity and the product selectivity are maintained well, and the formation of a polymeric by-product that causes process blockage is also suppressed.

### REFERENCE SIGNS LIST

1c Catalyst tank
10 Reactor
10a Stirrer
11, 22, 32, 41, 42, 51 pipe
11a Deactivator supply pipe
12 First supply pipe
12a Ethylene supply pipe
13 Second supply pipe
13a Catalyst supply pipe
14 Third supply pipe
15 Fourth supply pipe
21, 31 Circulation pipe
16 Condenser
17 Compressor
20 Degassing tank
30 Ethylene separation column
40 High-boiling separation column
50 Hexene separation column
52 Solvent circulation pipe
60 Solvent drum
1-HEX 1-Hexene
ETY Ethylene

1. A method for producing an α-olefin low polymer, the method comprising:
a gas drying step of supplying a gas into a reaction system;
a solvent drying step of supplying a drying agent-containing solvent containing a drying agent into the reaction system after the gas drying step;
a deactivator addition step of supplying a deactivator for the drying agent into the reaction system; and
an α-olefin low polymerization reaction step of performing a low polymerization reaction of an α-olefin,
wherein the deactivator is supplied into the reaction system prior to initiation of the low polymerization reaction of the α-olefin.

2. The method according to claim 1, wherein, in the gas drying step, the gas is supplied into the reaction system until a moisture content in the reaction system becomes equal to or less than a predetermined value (1).

3. The method according to claim 2, wherein the predetermined value (1) is 100 mol ppm.

4. The method according to claim 1, wherein a moisture content of the gas is 5 mol ppm or less.

5. The method according to claim 1, wherein, in the solvent drying step, the drying agent-containing solvent is supplied into the reaction system until a moisture content in the reaction system becomes equal to or less than a predetermined value (2).

6. The method according to claim 5, wherein the predetermined value (2) is 10 mol ppm.

7. The method according to claim 1, wherein the drying agent-containing solvent comprises an alkylaluminum compound.

8. The method according to claim 1, wherein the deactivator comprises an alkyl alcohol.

9. The method according to claim 1, further comprising, after the gas drying step and before the solvent drying step, a raw material replacement step of supplying a raw material α-olefin into the reaction system until a nitrogen gas content in the reaction system becomes equal to or less than a predetermined value (3).

10. The method according to claim 9, wherein the predetermined value (3) is 20 vol %.

11. The method according to claim 9, further comprising, after the raw material replacement step and before the solvent drying step, a solvent replacement step of supplying a reaction solvent into the reaction system until a content of the α-olefin in the reaction system becomes equal to or less than a predetermined value (4).

12. The method according to claim 11, wherein the predetermined value (4) is 70 vol %.

13. The method according to claim 1, further comprising, after the deactivator addition step and before the α-olefin low polymerization reaction step, a measurement step of measuring a moisture content in the reaction system,
wherein the α-olefin low polymerization reaction step is started when the moisture content measured in the measurement step is equal to or less than a predetermined value (5).

14. The method according to claim 13, wherein the predetermined value (5) is 10 mol ppm.

15. The method according to claim 1, wherein, in the α-olefin low polymerization reaction step, a catalyst, a solvent, and a raw material α-olefin are supplied into the reaction system.
